(19) United States
(12) Patent Application Publication
(10) Pub. No.: US 2026/0000889 A1
(43) Pub. Date: Jan. 1, 2026

(54) ORAL MUSCLE COMPOSITION DETECTION AND TRAINING

(71) Applicant: SIGNIFIER MEDICAL TECHNOLOGIES LIMITED, London (GB)

(72) Inventor: Akhil TRIPATHI, London (GB)

(21) Appl. No.: 18/328,670

(22) Filed: Jun. 2, 2023

Related U.S. Application Data
(63) Continuation of application No. PCT/GB2021/053153, filed on Dec. 2, 2021.

(30) Foreign Application Priority Data
Dec. 2, 2020 (GB) 2018997.3

(51) Int. Cl. A61N 1/36; A61F 5/56; A61N 1/02; A61N 1/05; G16H 20/30; G16H 40/63

(52) U.S. Cl. CPC A61N 1/3601; A61F 5/566; A61N 1/025; A61N 1/0548; G16H 20/30; G16H 40/63

(57) ABSTRACT

An apparatus for applying electrical stimulation to one or more muscles of the mouth of a user. The apparatus comprises one or more electrodes for applying electrical stimulation to one or more muscles of the mouth of a user. The apparatus also comprises a sensing assembly configured or configurable to determine or measure, in use, the tongue composition of the tongue of the user.

### CLAIM OF PRIORITY

This application is a continuation of International Application No. PCT/GB2021/053153, filed Dec. 2, 2021, titled “ORAL MUSCLE COMPOSITION DETECTION AND TRAINING,” now International Publication No. WO 2022/118028, which claims the benefit of United Kingdom Patent Application 2018997.3, filed Dec. 2, 2020 and titled “ORAL MUSCLE COMPOSITION DETECTION AND TRAINING,” each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The methods and apparatuses described herein relate generally to oral muscle training, particularly to oral muscle training devices, methods, systems, and control software. More specifically, although not exclusively, the methods and apparatuses described herein relate to devices and systems for determining the composition of the tongue, for example, for use in determining or modifying the parameters of a stimulation plan for the training of muscles of the mouth for the treatment of sleep disordered breathing.

### BACKGROUND

Snoring and sleep apnoea are considered as part of a range of conditions often termed as sleep disordered breathing (SDB), with symptoms relating to disordered breathing patterns during sleep. SDBs are not only a nuisance, but they can also result in health problems, for example frequent waking from sleep, light sleeping, strain on the heart, low oxygen levels in the blood, headaches and fatigue. Further SDB can also affect the sleeping partner of the symptomatic person by causing disturbed sleep.

The breathing passage of humans between the throat, back of the nose and mouth, to the level of the larynx, is a collapsible tube. It has been observed that collapse of the breathing passage occurs at a positive airway pressure in individuals who snore and/or suffer from sleep apnoea syndrome and at a negative airway pressure in individuals who do not.

In an effort to address this phenomenon, treatments have been developed which include using a continuous positive airway pressure device to keep the breathing passage open or wearing a mandibular advancement device to hold the jaw and tongue forward in order to increase the space at the back of the throat.

These devices can cause side effects such as jaw discomfort, a dry mouth and/or throat, air in the stomach, etc. These device may address the symptoms only temporarily, rather than addressing the underlying cause, and they must be used during sleep on an ongoing basis. As a result, users find it difficult to fall asleep or remain asleep and compliance is therefore low. Therefore, it would be desirable to provide a stimulation that does not require the regular use of such devices during sleep and that addresses the underlying cause or causes of the condition.

Recent research studies have shown that implanting electrical nerve stimulators into the tongue and diaphragm are effective in the treatment of obstructive sleep apnoea. This involves intrusive surgery to implant sensors and electrodes on nerves in these areas. The device identifies an episode of obstruction using the sensors and stimulates the tongue nerve to cause contraction to relieve the obstructive event. As with pacemakers, this approach leads to maintenance and other complications, such as battery replacement, risks associated with electrical fields and issues related to external security detection devices. In addition, stimulation only occurs during an obstructive episode during sleep; it does not address the underlying cause of the condition.

### SUMMARY OF THE DISCLOSURE

Described herein are methods and apparatuses that are configured both to detect muscle composition of one or more muscles of the oral cavity (e.g., tongue) and for applying electrical simulation to the one or more muscles. In some variations the method or apparatus may track the muscle composition over time, and/or may modify the electrical treatment applied based on the muscle composition or changes in muscle composition.

In examples, the muscle composition may comprise an indicator or indication of the anatomical make-up or structure of the muscle.

U.S. Pat. No. 9,833,613 describes one example of a device for the treatment of SDB configured to allow a user to train muscles of the mouth to improve muscle function (such as tone, responsiveness, etc.) and thereby stop, or at least inhibit, SDB events. The user may use the device to improve muscle function (such as tone, responsiveness, etc.). In one example the device has a mouthpiece for location between the upper and lower mandibular arches and a pair of flanges for engaging the upper or dorsal surface of the tongue additionally or alternatively there may be a pair of flanges for engaging the sublingual surface (underside) of the tongue, each of the flanges may include an electrode. Energising the device may cause an electric current to be applied to the tongue between the electrodes to improve tongue muscle function (such as tone, responsiveness, etc.). By muscle function we mean the physiological characteristics of muscle, for example the ability of muscle to produce force and/or to cause motion and/or to resist positional change, for example during relaxation.

The device disclosed in U.S. Pat. No. 9,833,613 has been clinically proven to reduce SDB events (e.g. Wessoleck et al. Somnologie (Berl)., 2018; 22(Suppl 2), 47-52 and Sama et al. CHEST Annual Meeting Notes, 2018). Furthermore, it has also been shown that it is possible to determine the parameters for therapy sessions (e.g. the current, frequency, and duration) for the development of a stimulation plan for a new patient, and/or for measuring the progress of an ongoing stimulation plan of a current patient. This is performed by using measurements taken by a sensor for determining the muscle function (such as tone, responsiveness, etc.) of the tongue of the user.

A stimulation plan may comprise a number, for example a set number, of stimulation sessions (e.g. plural stimulation sessions). During a stimulation session, a device or portion of a device, may be inserted into the patient's mouth and the device may be energised for a period of time. Each stimulation session within the stimulation plan may comprise or consist of one or more parameters that may be fixed or variable during or between stimulation sessions. Such parameters may include one or more of the stimulation time, and/or the intensity (amplitude) of the current delivered by one or more electrodes on the device, the frequency of the applied current, pulse width, pulse duration, type of current, the current application time, continuous or bursts of current. If the device comprises more than one electrode or set of electrodes, for example, a first set that engages the dorsal surface of the tongue, and a second set that engages the sublingual surface of the tongue, it may also be possible that each set of electrodes delivers, for example a different current, different intensity of electric current, different frequency of current and so on. Further, the direction of stimulation (vertical, e.g. sublingual to dorsal, lateral, e.g. sublingual to sublingual, or vertically diagonal, e.g. right dorsal to left sublingual) may be altered one or more times during a stimulation session or between successive stimulation sessions within a stimulation plan.

The treatment requirements for the use of such a device may vary from patient to patient. Consequently, it may be advantageous that a device enables the development of one or more tailored stimulation sessions within a stimulation plan, according to the user's status at the beginning of a stimulation plan. It may also be advantageous for the device to also enable measurement of the progress and/or the efficacy of a stimulation plan, such that the parameters of one or more future stimulation sessions may be tailored to the user's progress during the stimulation plan and/or responsiveness to previous stimulation sessions within the stimulation plan.

It may be further advantageous to be able to gather further information on the relative health or status of the tongue muscle of the patient. This could be used to further inform parameters for therapy sessions for the development of a stimulation plan for a new patient, and/or for measuring the progress of an ongoing stimulation plan of a current patient.

Thus, described herein are apparatuses for applying electrical stimulation to one or more muscles of the mouth and/or tongue of a user, in some examples to be used in an awake state, that may enable measurements of initial patient status and/or patient progress on a stimulation plan to be performed.

Accordingly, described herein are apparatuses for applying electrical stimulation to one or more muscles of the mouth of a user, the apparatus comprising one or more electrodes for applying electrical stimulation to one or more muscles of the mouth of a user and a sensing assembly configured or configurable to determine or measure, in use, the tongue composition of the tongue of the user.

Whilst determining the muscle function provides an indication of a user's progress during a stimulation plan, for example an increase in strength resulting from improved muscle tone, muscle function does not provide information about the composition and/or status, e.g. health, of the tongue muscle or muscles. Determining the tongue composition may advantageously provide further, for example more detailed, information regarding the status of the tongue muscle or muscles.

It has been surprisingly found that measurements taken from the sensing assembly may be converted into measurements which are at least representative of tongue composition. Beneficially, the user may generate a stimulation plan, monitor the progress of their stimulation plan, and/or adjust their stimulation plan according to the measurements taken by the sensor.

In examples the tongue composition may comprise an indicator or indication of the anatomical make-up or structure of the tongue.

Determining or measuring the tongue composition of the tongue of the user may include determining or measuring the volumetric or mass ratio of muscle to fat of the tongue of the user. As such, the apparatus may be operable to perform a quantitative analysis of the tongue of the user. In some examples, the sensing assembly may be configured to determine or measure the tongue composition by determining or measuring a volumetric or mass ratio of muscle to fat of the tongue of the user.

In some examples, determining or measuring the tongue composition of the tongue of the user may comprise determining or measuring the total volume or mass of muscle and/or fat of the tongue of the user. In some examples, the sensing assembly may be configured to determine or measure the tongue composition by determining or measuring the total volume or mass of muscle and/or fat of the tongue of the user. In examples, determining or measuring the tongue composition of the tongue of the user may comprise determining or measuring the percentage volume or mass of muscle and/or fat of the tongue of the user.

Determining or measuring the tongue composition of the tongue of the user may include determining or measuring the mass or volume of one or more types of muscle of the tongue of the user. In some examples, the sensing assembly may be configured to determine or measure the tongue composition by determining or measuring the mass or volume of one or more types of muscle of the tongue of the user. In some examples, determining or measuring the tongue composition of the tongue of the user may comprise determining or measuring the relative ratio (e.g. or percentage) of a first type of muscle to a second type of muscle of the tongue of the user. In some examples, the sensing assembly may be configured to determine or measure the tongue composition by determining or measuring the relative ratio (e.g. or percentage) of a first type of muscle to a second type of muscle of the tongue of the user. A first type of muscle may be a slow-twitch muscle. A second type of muscle may be a fast-twitch muscle.

In some examples, determining or measuring the tongue composition of the tongue of the user may comprise determining or monitoring the or a change in overall tongue volume. In some examples, determining or measuring the tongue composition of the tongue may comprise determining or measuring the composition of the genioglossus muscle.

In some examples, the sensing assembly may comprise a sensor. The sensing assembly may comprise more than one sensor, e.g. two, three, four, five, six, or more sensors, for example, each of which may be usable to determine the composition of the tongue of the user. In examples comprising more than one sensor, each sensor may be usable to determine the same parameter. In alternative examples comprising more than one sensor, one, some or each sensor may be usable to determine a different parameter, for example a different tongue composition parameter. The parameter(s) may comprise: the mass of muscle, the volume of muscle, the mass of fat, the volume of fat, the volume ratio of fat to muscle, the mass ratio of fat to muscle, the percentage by mass of fat, the percentage by mass of muscle, the percentage by volume of fat, the percentage by volume of muscle, the overall volume of the tongue, the volume or mass of a first type of muscle, the volume or mass of a second type of muscle, the ratio of the first to second type of muscle.

In some examples, the, one, some or each sensor may be selected from one or more of an impedance sensor, a temperature sensor, or an electromyography (EMG) sensor.

In some examples, the, one, some or each sensor may be an impedance sensor configured to measure the impedance at different locations on the tongue in response to an applied current. In an example, the one or more electrodes of the apparatus may be used as one or more sensors, for example to monitor or determine impedance of the tongue. In other examples, the impedance measurements may be affected using one or more additional or dedicated electrodes.

Where the, one, some or each sensor comprises an impedance sensor the sensing assembly may be configured to evaluate the tongue composition at a single (e.g. specific) frequency (e.g. over time), for example about 50 kHz (e.g., above 0 kHz, say about 1 kHz, about 5 kHz, about 10 kHz, about 15 kHz, about 20 kHz, about 25 kHz, about 30 kHz, about 35 kHz, about 40 kHz, about 45 kHz, about 50 kHz, about 55 kHz, about 60 kHz, about 65 kHz, about 70 kHz, about 75 kHz, about 80 kHz, about 85 kHz, about 90 kHz, etc.), over a duration of, e.g., about 2 minutes (e.g., about 1 min, about 2 min, about 3 min, about 4 min, about 5 min, about 6 min, about 7 min, about 8 min, about 9 min, about 10 min, etc.) or less. For example, the one or more impedance sensor(s) may be used to evaluate the tongue composition at a single (e.g. a specific) frequency, for example over time (e.g. about 50 kHz, say over about 2 minutes, etc.).

Where the, one, some or each sensor comprises an impedance sensor the sensing assembly may be configured to evaluate the tongue composition at multiple frequencies, for example over a range of frequencies (e.g. between about 0 to 100 kHz). The one or more impedance sensors may be used to evaluate the tongue composition at multiple individual or groups of frequencies, for example, over a range of frequencies (e.g. between 0 to 100 kHz). The one or more impedance sensors may be used to evaluate the tongue composition at multiple individual or groups of frequencies over time, for example during a sampling window (e.g. between 0 to 90 seconds or longer). The sampling window may be about 20 seconds, about 30 seconds, about 40 seconds, about 50 seconds, about 60 seconds, about 70 seconds, about 80 second, about 90 seconds, etc.

The sensing assembly may comprise an impedance converter, for example configured or configurable to convert impedance data received from the sensor into useable data (for example into user readable data). An example of an impedance converter is the AD5933, available from Analog Devices Inc.

The apparatus and/or the sensing assembly may be configured to perform a bioimpedance analysis of the tongue or a portion thereof. The apparatus and/or the sensing assembly may be configured to perform a single-frequency bioelectric impedance analysis (SF-BIA), for example of the tongue or a portion thereof. The apparatus and/or the sensing assembly may be configured to perform a multi-frequency bioelectric impedance analysis (MF-BIA), for example of the tongue or a portion thereof.

The sensing assembly may be configured or configurable to perform bioelectrical impedance vector analysis (BIVA) to determine the composition of the tongue of the user. BIVA is a non-invasive method of measuring human body composition, e.g. the composition of the tongue. The, one, some or each impedance sensor and/or the impedance converter may be usable to perform bioelectrical impedance vector analysis to determine the composition of the tongue of the user.

In some examples, the sensing assembly may be configured or configurable to perform electrical impedance tomography (EIT), for example using impedance data from the, one, some or each impedance sensor (where provided). Electrical impedance tomography (EIT) is a non-invasive type of medical imaging in which the electrical conductivity, permittivity, and impedance of a part of the body is inferred from surface electrode measurements and used to form a tomographic image of that part. The difference in electrical conductivity between the fatty tissue and the muscle mass of the tongue is differentiated by application of small alternating currents at a single frequency or using multiple frequencies (e.g. a range of frequencies).

In some examples, one or more of the sensors may be a temperature sensor configured to measure the temperature of specific locations of the tongue. The temperature sensor may comprise or be a thermistor. It is known that vasodilation occurs naturally in the body in response to triggers such as low oxygen levels, a decrease in available nutrients, and increases in temperature. When thermoreceptors pick up on a higher amount of warmth in an environment relative to cold, vasodilation will occur. This directs a higher flow of blood toward the skin to dissipate any excess warmth felt. The opposite is true for vasoconstriction. Without wishing to be bound by any particular theory, it is believed that electrical stimulation of oral muscles during a stimulation plan causes the muscle mass to increase, which causes a change in the muscle fibre composition. This has an impact on the perfusion of one or more tongue muscle, and on vasodilatation or vasoconstriction. Therefore, the structure of muscle mass in comparison to fat content generates a different temperature, which is sensed by the sensors of the methods and apparatuses described herein.

In some examples, one or more of the sensors may be an EMG (electromyograph) sensor configured to record EMG signals from one or more locations on the tongue. EMG signals are recorded by detecting the potential generated by a muscle during contraction.

The sensor or one or more sensors may be passive, requiring no conscious interaction by the user, or active, where the user performs one or more specific interactions with the sensor(s) and/or apparatus. Advantageously, the use of one or more impedance, temperature, or EMG sensors to measure tongue composition of one or more oral muscles is a passive process. That is, the user does not need to actively interact with the apparatus in order for the sensing assembly to determine the tongue composition of the user.

In some examples, the apparatus may be for use in training oral muscle function (e.g. tone, responsiveness, etc.). The apparatus may be configured to provide, in use, via the one or more electrodes electrical stimulation to one or more oral muscles, e.g. tongue muscle and optionally palate muscles, through the lining of the mouth, for example the oral mucosa, e.g. to increase resting muscle function (e.g. tone, responsiveness, etc.) and/or muscle function (e.g. tone, responsiveness, etc.) during sleep.

In other examples, the apparatus may be used to determine or monitor the tongue composition as part of a health assessment for a specific clinical condition, e.g. Amyotrophic Lateral Sclerosis (ALS), Type II Diabetes, or oral cancers (e.g. OSCC, Oral Squamous Cell Carcinoma/OPMDs, Oral Potentially Malignant Disorders).

The sensing assembly may comprise electrical circuitry operatively connected to the one or more electrodes. The sensing assembly may comprise electrical circuitry operatively connected to the sensor or one or more or each of the sensors.

Advantageously, the sensing assembly may be configured to provide feedback information to the user on changes, e.g. improvements, to the composition of their tongue. For example, the changes or improvements to the composition of their tongue may comprise a change (e.g. an increase or reduction) in fat or muscle mass or volume, or a change (e.g. increase or reduction) in the relative ratio of fat to muscle mass or volume in the tongue.

In some examples, the sensing assembly may include a sensing system to determine or monitor the composition of the tongue before, during and/or subsequent to a treatment session or treatment plan.

The sensing system may comprise an impedance sensing system. In some examples, the impedance sensing system may be a system for performing electrical impedance tomography. In some examples, the impedance sensing system may be usable to provide an image of the tongue, for example, a cross-sectional image (e.g. a tomogram) of the tongue.

It has been found that treatment causes significant changes in the impedance of the tongue and thus the resistance, reactance and phase shift of stimulation (e.g. phase shift of stimulation pulses) can be determined. These measurements can be used to determine the composition of the tongue and may be used or be usable to ‘image’ the tongue and thus may be able to provide a dynamic visualisation or graphical representation of progress.

This information may be used to recommend further treatment sessions, regimes, and/or plans and/or to alter or amend existing treatment sessions, regimes and/or plans.

Research has demonstrated that increasing the pharyngeal muscle activity reduces the collapsibility of the airway. The methods and apparatuses disclosed in U.S. Pat. No. 9,833,613 is based on the realisation that electrical stimulation, particularly neuromuscular electrical stimulation, can be used to stimulate the muscles of the tongue and/or palate and/or the sensory nerves to improve muscle power and tone recovery.

When a person is awake, the collapsible segment of the breathing passage is kept open due to the muscles that control this area. When a person is asleep, this muscle function (e.g. tone, responsiveness, etc.) reduces significantly. Evidence has shown that a reduction of muscle mass and/or an increase of fat content (and the consequent increase in the relative ratio of fat content in the tongue) is significantly greater in individuals who suffer from obstructive sleep apnoea, less so in those who snore and notably less in individuals who suffer from neither of these disorders.

The present methods and apparatuses described herein provide a sensing assembly which is able to measure or determine the composition of the tongue of the user. Beneficially, the user and/or another (for example a healthcare professional) is able to monitor the progress of the user's stimulation plan. By using the data generated for the composition of the tongue, the stimulation plan can be modified in response to measured data. For example, a processor (e.g. comprised of the apparatus or the sensing assembly) can determine progress or change in the composition of the tongue compared to an expected or predicted composition (e.g. a predicted or desired fat to muscle ratio) and alter the stimulation plan accordingly. Additionally or alternatively, the change in tongue composition (e.g. decrease in fat content and/or increase in muscle mass) might be communicated to the user (or another) for example visually, aurally or otherwise.

In some examples, data relating to tongue composition may be provided to a computer software program (for example an APP held on a computing device) for display of tongue composition data, for example showing changes in tongue composition over time.

An aspect of the methods and apparatuses described herein comprises a system comprising an apparatus as described herein and a computing device, for example containing or programmed with said computer software program.

The or a computer software program may be able to process received data relating to the composition of the tongue, for example to modify a stimulation plan.

For example, if changes in the tongue composition of the user are not progressing as expected given the stimulation plan, the or a computer software program may be configured to adjust, e.g. automatically adjust, the stimulation plan, for example by increasing one or more of the characteristics of the electrical stimulation (intensity, current, duration and so on). In this way the apparatus may be configured to improve the tongue composition, e.g. to reduce the quantity or relative ratio of fat, and/or to increase the quantity or relative ratio of muscle. Conversely, if the user's tongue composition is improving more rapidly than expected given the stimulation plan the or a computer software program may be configured to adjust, e.g. automatically adjust, the stimulation plan, for example by decreasing one or more of the characteristics of the electrical stimulation (intensity, current, duration and so on). Advantageously, the automatic adjustment of the stimulation plan does not require a doctor or physician's input.

Additionally or alternatively, the user may manually select a new stimulation plan, for example which selection may be based on the change in tongue composition data.

In some examples, a computer software program on a computing device (e.g. an APP held on a portable computing device) is operable to recommend a future stimulation plan, and/or modifications to stimulation sessions within an existing stimulation plan, in response to the user requirements and/or the progress on a current stimulation plan. For example, the recommendations may include to increase or decrease the intensity of the electric current being supplied by one or more electrodes on the apparatus. Additionally or alternatively, the recommendations may include to increase or decrease the set time of one or more therapy sessions within a stimulation plan.

The apparatus and/or the sensing assembly may further comprise a controller (or control means, e.g. a control unit) which may be programmed or programmable, for example, to activate and/or control the sensing assembly or the, one, some or each sensor. The controller (or control means, e.g. a control unit) may be programmed or programmable, for example, to activate and/or control the electrodes. The controller (or control means, e.g. a control unit) may be operable to alter the electrical stimulation to said one or more muscles of the user's mouth, e.g. in use.

The controller (or other control means, e.g. a control unit) may comprise one or more dials for varying the output of the apparatus, and/or for initiating or halting a test event, e.g. a tongue composition measurement event. The controller (or control means, e.g. a control unit) may interface with, for example, the or a computer software program held on the or a computing device, for example the or an APP held on a mobile device, such as a personal computer, smart phone or tablet. The computer software program may be programmed to conduct a measurement of a parameter, e.g. the relative ratio of fat content to muscle mass in the tongue of the user. The computer software program may be programmed to conduct a measurement of a parameter, e.g. the impedance, the temperature, and/or EMG signals. The controller (or control means, e.g. a control unit) may comprise a control system and/or may comprise or be at least partially comprised in the electrical circuitry.

In some examples, the controller (or control means, e.g. a control unit) may be for or be configured or operable to control and/or adjust one or more parameters of measurements taken by the sensor or one or more sensors, for example, measurement time for measuring the tongue composition of the user.

The sensing assembly or controller (or control means, e.g. a control unit) may be configured or programmed to control one or more features of the tongue composition measurement. The sensing assembly or controller (or control means, e.g. a control unit) may be operable or programmed to create and/or alter, e.g. to automatically create and/or alter, the predetermined stimulation regime, for example by a device to which the apparatus is connected, e.g. in response to the data collected by the sensor or one or more sensors on the tongue composition of the user.

The apparatus may comprise a switch means or switch operable to transform the apparatus from stimulation mode, whereby a stimulation session may be provided by the apparatus, to a test mode (and vice versa), whereby the tongue composition of the user can be assessed. The switch means or switch may be manually or automatically controlled, e.g. by the controller (or control means, e.g. a control unit). Said switch means or switch may be operable directly, for example by the user causing a switch to switch on the apparatus, or remotely, for example by accessing a computing device linked to the apparatus (e.g. via an APP on a personal computing device such as a PC, smartphone, tablet and so on).

In some examples, the sensing assembly may comprise a frequency adjuster or generator, e.g. configured or configurable to adjust or generate a or the frequency of electrical energy supplied to the electrodes.

In some examples, the, one, some or each sensor may be mounted onto, or into, the apparatus, e.g. releasably or permanently mounted onto or into the apparatus.

The apparatus may comprise a mouthpiece for locating in a user's mouth. The mouthpiece may have or comprise one or more electrodes for applying electrical stimulation to one or more muscles of the mouth of a user associated with the mouthpiece.

In some examples, the mouthpiece may comprise one or more arms and/or one or more appendages or flanges which may extend from the one or more arms, e.g. for contacting one or more oral muscles. At least one arm and/or at least one appendage or flange may be flat or planar, for example with major surfaces. Optionally, the mouthpiece may comprise a pair of arms each of which may comprise one or more appendages or flanges. In some examples, the mouthpiece may comprise a pair of arms that may extend at least partially alongside each other and/or at an angle relative to one another and/or parallel to each other. For example, the mouthpiece may comprise a pair of arms connected together at a connecting portion, for example and extending away from one another. In some examples, the mouthpiece may comprise flanges for overlying at least a portion of the dorsal or sublingual surface of the user's tongue. In some examples, the mouthpiece may comprise a pair of arms connected together at one end and diverging from one another, for example in a substantially V-shape or U-shape or horseshoe shape.

The one or more appendages or flanges may extend inwardly of the pair of arms, e.g. from one arm and toward the other arm. In some examples, each arm comprises at least one appendage or flange, for example opposite one another and/or extending toward one another. In some examples, each arm comprises two or more appendages or flanges, for example an appendage or flange extending from a free end of each arm and/or an appendage or flange extending from an intermediate portion of each arm.

At least one appendage or flange may be curved, e.g. a flat curved shape or member, and/or extend upwardly or downwardly or out of the plane of the mouthpiece or at least one arm thereof. At least one appendage or flange may be shaped to cooperate or approximate or accommodate a tongue surface, for example a dorsal tongue surface or a sublingual tongue surface. In some examples, the mouthpiece comprises at least one appendage or flange that is shaped to cooperate or approximate or accommodate a dorsal tongue surface and at least one appendage or flange that is shaped to cooperate or approximate or accommodate a sublingual tongue surface. In some examples having a pair of arms, each arm may comprise an appendage or flange shaped to cooperate or approximate or accommodate a dorsal tongue surface and an appendage or flange that is shaped to cooperate or approximate or accommodate a sublingual tongue surface.

At least one of the appendages or flanges may comprise one or more electrodes or series thereof. At least one electrode or series of electrodes may be adjacent and/or associated with and/or exposed at a surface, e.g. a major surface, of the at least one appendage or flange. In some examples, at least one of the appendages or flanges comprises electrodes associated with each of its major surfaces. The electrodes associated with one of the major surfaces may be isolated and/or controllable independently from another or the other major surface thereof. Additionally or alternatively, the electrodes of or associated with one appendage or flange may be isolated and/or controllable independently from at least one other appendage or flange.

In some examples, the mouthpiece may comprise a pair of arms joined together at one end and diverging from one another to provide a substantially horseshoe shape with one or more flanges extending inwardly from at least one arm, the or each flange comprising an electrode. The mouthpiece may comprise a pair of flanges each extending inwardly from a respective arm, which flanges are shaped to accommodate a dorsal tongue surface. The mouthpiece may comprise a pair of flanges each extending inwardly from a respective arm, which flanges are shaped to accommodate a sublingual tongue surface. Each of the pair of flanges may be shaped to accommodate a dorsal tongue surface extends from at or adjacent a free end of the arm and/or each of the pair of flanges may be shaped to accommodate a sublingual tongue surface extends from an intermediate portion of the arm. In an example each of said first and second arms may have a longitudinal axis and comprise a flange extending as a continuation of the longitudinal axis from a free end of the arm, each flange preferably carrying said at least one electrode.

In some examples, the sensor or one or more sensors, e.g. an impedance sensor, a temperature sensor, an EMG sensor; may be located or locatable on the mouthpiece. In some examples, the sensor or one or more sensors may be located or locatable, where present, on one or more of the flange(s). For example, a first sensor, e.g. an impedance sensor, a temperature sensor, an EMG sensor; may be located or locatable on a first flange that is shaped to accommodate the dorsal surface of the tongue, and/or a second sensor, e.g. an impedance sensor, a temperature sensor, an EMG sensor; may be located or locatable on a second flange that is shaped to accommodate the dorsal surface of the tongue. Additionally or alternatively, a first sensor, e.g. an impedance sensor, a temperature sensor, an EMG sensor; may be located or locatable on a first flange that is shaped to accommodate the sublingual surface of the tongue, and/or a second sensor, e.g. an impedance sensor, a temperature sensor, an EMG sensor; may be located or locatable on a second flange that is shaped to accommodate the sublingual surface of the tongue. In some examples, one or more sensor(s) is located proximal or adjacent to the one or more electrodes.

Additionally or alternatively, the, one, some or each sensor may be located or locatable on one of, or both of, the pair of arms of the mouthpiece. For example, one or more sensor may be located on one of the pair of arms and one or more other sensor may be located on the other of the pair of arms.

Another aspect of the methods and apparatuses described herein provides an electrical stimulation device for training one or more oral muscles, for example a trans mucosal neuromuscular electrical stimulation device, the device comprising a mouthpiece, electrode associated with the mouthpiece and electrical circuitry operatively connected to the electrode, wherein the mouthpiece comprises a pair of arms joined together at one end and diverging from one another with one or more flanges extending inwardly from at least one arm, the or each flange including at least part of the electrode associated therewith for providing electrical stimulation to one or more oral muscles, the electrical stimulation device further comprising a sensor for determining or measuring the composition of the tongue of the user.

Yet another aspect of the methods and apparatuses described herein provides an electrical stimulation mouthpiece for training one or more oral muscles, for example a trans mucosal neuromuscular electrical stimulation mouthpiece, the mouthpiece comprising a pair of arms joined together at one end and diverging from one another with one or more flanges extending inwardly from at least one arm, wherein the or each flange includes electrode associated therewith for providing electrical stimulation to one or more oral muscles, the electrical stimulation device further comprising a sensor for determining or measuring the composition of the tongue of the user.

For the avoidance of doubt, any of the features described herein apply equally to any aspect of the methods and apparatuses described herein.

The apparatus is preferably configured to provide a current, for example an electric current or impulse current, which may be selected from one or more of a Russian current, interferential current, pre-modulated current, DC electric current, biphasic electric current or impulse current. Other current forms may be used.

By providing a biphasic electric current, particularly a biphasic electric impulse current, tongue and/or palate muscles contributing to the collapsibility of the airway can be stimulated along with the sensory nerves to increase resting muscle function (e.g. tone, responsiveness, etc.) and muscle function (e.g. tone, responsiveness, etc.) during sleep.

The current is preferably a biphasic symmetrical current, but it may additionally or alternatively be a biphasic asymmetrical current that may either be balanced or unbalanced. The apparatus or electrical circuitry may be configured to provide, in use, via the electrodes an electric current with a frequency of up to 150 Hz, say between 1, 2, 3, 4 or 5 Hz and 150 Hz.

The electric current may comprise a frequency of between 10 and 140 Hz, for example between 15 and 130 Hz, preferably between 20 and 120 Hz. Preferably, the electric current comprises a frequency of between 1 and 50 Hz and/or between 2 and 120 Hz.

The apparatus or electrical circuitry may be configured to provide two or more currents, for example a first current and/or a second current, which second current may be different from and/or configurable or settable independently from the first current. At least one, e.g. both, of the first and/or second current may comprise a biphasic (or other) current, each of which is preferably symmetrical, but may be asymmetrical and either balanced or unbalanced. At least one of the first and/or second current may comprise a frequency of between 1, 2, 3, 4, 5 and 150 Hz, for example between 1 and 140 Hz, e.g. between 1 and 130 Hz, preferably between 2 and 120 Hz. In some examples, one or the currents may comprise a frequency of between 2 and 50 Hz and/or the other current may comprise a frequency of between 1 and 120 Hz.

The inventors believe that the application of an electric current in one or each of these two frequency ranges is particularly suited to targeting tongue muscles contributing to the collapsibility of the airway.

The apparatus or electrical circuitry may be configured to provide, in use, the or at least one or each electrical current to one or more oral muscles, such as palate and/or tongue muscles, for example through the lining of the mouth, e.g. the oral mucosa, such as to increase resting muscle function (e.g. tone, responsiveness, etc.) and/or muscle function (e.g. tone, responsiveness, etc.) during sleep. In some examples, the apparatus is configured to provide, in use, the electrical current, e.g. the first electrical current, to one or more palate muscles. Additionally or alternatively, the apparatus may be configured to provide, in use, the electrical current, e.g. the second electrical current, to one or more tongue muscles, e.g. via the dorsal tongue surface. Additionally or alternatively, the apparatus may be configured to provide, in use, the electrical current, e.g. the first or second electrical current, to one or more tongue muscles via the underside of the tongue.

The mouthpiece (where provided) may comprise a gripping base, which may comprise an enlarged end, e.g. an enlarged free end, which may be connected or secured to, e.g. formed integrally with, the mouthpiece or a body or one or more or each arm thereof, for example by a necked portion.

The electrodes preferably comprise at least one anode and at least one cathode, for example two or more anodes and/or two or more cathodes, e.g. a plurality of anodes and a plurality of cathodes. At least part of the electrodes, for example one or more or each or all of the electrodes, may be mounted to or on or within and/or at least partially housed or contained within the mouthpiece. In some examples, the mouthpiece comprises a shield (e.g., shield means), for example on one side of the electrodes, e.g. for inhibiting or preventing the electrical stimulation or current from being applied or provided by or at or from one side of the mouthpiece. Suitable materials for the shield (for example shield means) will be apparent to those skilled in the art.

In some examples, the electrodes are configured or operable to provide or apply, e.g. selectively, the electrical stimulation or current at or from at least one or each or both sides, for example by including shielding (e.g., a shield or shield means) between a first set or series of electrodes and a second set or series of or electrodes. In some examples, the first electrical current is provided or applied at or from a first side, e.g. major side, of the mouthpiece and/or by the first set or series of or electrodes. Additionally or alternatively, the second electrical current may be provided or applied at or from a second side, e.g. major side, of the mouthpiece and/or by the second set or series of electrodes. In other examples, the first and second electrical currents may be provided or applied from at least one or each or both sides.

The mouthpiece may be insertable into the mouth and held in place, e.g. manually. The mouthpiece may be at least partially flattened and/or substantially flat and/or paddle-shaped, for example with at least one flat and/or major surface, preferably two flat major surfaces. In some examples, the apparatus may include a handle to which the mouthpiece may be connected or mounted or attached, for example rigidly and/or releasably, e.g. to enable the mouthpiece to be inserted and/or held, in use, within one or more locations or positions and/or orientations within the mouth. In some examples, the mouthpiece is free of any mount (e.g., a mounting means) for mounting or securing it to or in or within the mouth of a user.

In some examples, the mouthpiece may include a mount (e.g. mounting means). The mount (e.g. mounting means) may be for mounting the mouthpiece to an upper part or portion of the mouth, for example such that the mouthpiece or the or a first side or surface thereof is or may be in contact with and/or adjacent one or more palate muscles and/or the roof of the mouth and/or the mouthpiece or the or a second side or surface thereof is or may be in contact with and/or adjacent one or more tongue muscles, for example a dorsal tongue surface. Additionally or alternatively, the mount (e.g. mounting means) may be for mounting the mouthpiece to a lower part or portion of the mouth, for example such that the mouthpiece or the or a first side or surface thereof is or may be in contact with and/or adjacent one or more tongue muscles, for example a sublingual tongue surface. In some examples, the apparatus comprises a first mouthpiece with a mount (e.g. mounting means) for mounting it to an upper part or portion of the mouth and a second mouthpiece for mounting it to a lower part or portion of the mouth.

The apparatus may comprise an input means (e.g., an input or activator), which may include one or more input devices, buttons and/or push buttons and/or switches and/or dials or the like, e.g. for enabling or activating or initiating the electrical stimulation or current, or for enabling or activating or initiating measurement of the tongue composition of the user. The apparatus may comprise a power source and/or a cable connectable to a power source. In some examples, the apparatus comprises a main body that includes or incorporates or provides the mouthpiece or handle and/or which includes or houses the power source, which may comprise a rechargeable power source or one or more batteries that may be rechargeable, and/or which can either include the cable or be operatively, e.g. inductively, connectable to a charging station that includes or incorporates the cable, for example to enable the power source to be recharged. The apparatus may include the charging station.

The apparatus may comprise a memory means (e.g., a memory, such as non-volatile memory, flash memory, semiconductor memory, etc.), for example on which is stored a database for the conversion of data from the sensor into tongue composition data. The memory means (e.g. a memory) may be to hold data relating to electrical stimulation applied to said one or more muscles of the user and/or data relating to the output of the sensing assembly.

The apparatus may comprise a processing means, e.g. a processor, (for example which may be operatively connected to the sensor of the apparatus and to the memory means, e.g. the memory). The processing means or processor may be to process data relating to electrical stimulation applied to said one or more muscles of the user and/or data relating to the output of the sensing assembly.

The apparatus or sensing assembly may be configured, on or after a measurement event, to determine the tongue composition of the user, for example by comparing the data from the sensor with the data within the database of the memory, using the processor.

A further aspect of the methods and apparatuses described herein provides a system for measuring the tongue composition of a user, the system comprising: a mouthpiece held between the teeth of a user; a sensor for measuring the tongue composition in or on or associated with mouthpiece; optional memory or memory means for holding data received from the sensor; a processor or processing means to process data received from the sensor. The mouthpiece preferably comprises electrodes for stimulating one or more muscles of the mouth. The processor or processing means may be operable to control and/or adjust the output of the electrodes based on the data received from the sensor or one or more sensors. A yet further aspect of the methods and apparatuses described herein provides a system for measuring the tongue composition of a user, the system comprising: a mouthpiece, e.g. an electrical stimulation device, for training oral muscle function (e.g. tone, responsiveness, etc.), the device comprising a mouthpiece having at least one electrode associated with the mouthpiece, electrical circuitry operatively connected to the electrode, wherein the device is configured to provide, in use, via the at least one electrode electrical stimulation to one or more oral muscles, e.g. tongue muscle and optionally palate muscles, through the lining of the mouth, for example the oral mucosa, e.g. to increase resting muscle function (e.g. tone, responsiveness, etc.) and/or muscle function (e.g. tone, responsiveness, etc.) during sleep; a memory or memory means on which is stored a database for the conversion of data from the sensor into tongue composition data; a processor or processing means operatively connected to the sensor of the device and to the memory or memory means; wherein the system is configured, on or after a measurement event, to determine the tongue composition of the user, by comparing the data from the sensor with the data within the database of the memory or memory means, using the processor or processing means.

In some examples, the sensor, for measuring the tongue composition of the one or more oral muscles in or on or associated with a device, comprises one or more impedance sensors, temperature sensors, or EMG sensors, e.g. for measuring impedance, temperature, or EMG signals respectively. The memory or memory means may comprise a database suitable for the conversion of data, e.g. impedance, temperature, or EMG signal data, from the sensor, e.g. the one or more impedance sensors, temperature sensors, EMG sensors, into data, e.g. user readable data, on the tongue composition of the user. The processor or processing means may be configured to convert data, e.g. impedance, temperature, or EMG signal data from one or more impedance sensors, temperature sensors, or EMG sensors, into data, e.g. user readable data, on the tongue composition of the one or more oral muscles of the user.

In some examples the sensor may be operable to monitor the progressive characteristic changes in tongue composition. Such changes in structure may be captured by methods such as electrical impedance tomography using the electrodes used in stimulation and/or a dedicated one or more electrodes.

Advantageously, the system or apparatus of the methods and apparatuses described herein enables the user to be able to measure the tongue composition of the user, via a sensor, e.g. one or more impedance sensors, or one or more temperature sensors, or one or more EMG sensors.

The or a database of the or a memory or memory means (or at least accessible by said memory or memory means) may further comprise values for ‘normal’ or ‘healthy’ tongue composition data, e.g. according to the rest of the population and/or a model heathy user who does not suffer from sleep apnoea. The apparatus or system may enable the user and/or a healthcare professional to compare the tongue composition data or measurement of the user with ‘normal’ or ‘healthy’ tongue composition data stored within the database. The apparatus or system may enable the user and/or healthcare professional to design a future stimulation plan based on the comparison of data between the user and the values for ‘normal’ or ‘healthy’ tongue composition data.

The database of the memory or memory means may be capable of storing data, e.g. data from previous tongue composition measurement events performed by the user. The database of the memory or memory means may further comprise stored data from previous tongue composition measurement events performed by the user. The apparatus or system may enable the user and/or healthcare professional to perform comparisons between the tongue composition data of the user over time. For example, the apparatus or system may enable the user and/or healthcare professional to compare the tongue composition data or measurement of the user from a second tongue composition measurement event, with the tongue composition data or measurement of the user from a first tongue composition measurement event. The apparatus or system may enable the user and/or healthcare professional to modify or tailor the therapy session within a stimulation plan based on their progress, which is established through comparison of the user's tongue composition data at specific tongue composition measurement events over time.

The database of (or accessible by) said memory or memory means may further comprise data for the projected or targeted progress of the user on a stimulation plan based on the parameters, e.g. therapy session time, electric current intensity, electric current intensity of one or more specific electrodes, and so on, of the therapy sessions within the stimulation plan. The apparatus or system may be configured to retrieve data from the database and compare the user's data from two or more previous tongue composition measurement events performed by the user with the projected or targeted progress expected on that stimulation plan, such that the system is able to make recommendations on the parameters of future therapy sessions.

Advantageously, the apparatus or system is able to track the progress of the user's stimulation plan such that the healthcare professional is able to modify or tailor the therapy sessions within the treatment plan. More advantageously, the apparatus or system may be able to compare projected or targeted progress with actual progress, such that the system is able to make recommendations on the parameters for future therapy sessions within a treatment plan.

The apparatus or system may further comprise an alert means or mechanism (e.g. an alert), for example to alert the user that the apparatus or a part thereof has been incorrectly located within their mouth, and/or that the apparatus or system is unable to perform a measurement using the sensor. The alert or alert means or mechanism may comprise an audible or visual alert or device (e.g. an audible or visual alert means or mechanism). The alert means or alert or mechanism may comprise an audible means (e.g., speaker), e.g. for providing an audible alert or indication or statement or description, e.g. that the apparatus or a part thereof has been incorrectly located within the mouth of the user, and/or a display means or display, e.g. for providing a visual representation, e.g. that the apparatus or a part thereof has been incorrectly located within the mouth of the user.

The apparatus or system may further comprise a data transfer means or element or module or component or device, for example a port, e.g. a USB or serial port, or a wireless transmitter, e.g. a radio or Bluetooth or Wifi transmitter, for transferring data from at least one of the memory or memory means for review or analysis. Additionally or alternatively, the apparatus or system may comprise a display for displaying data stored in or on at least one of the memory or memory means.

The apparatus or system may further comprise a communication means, e.g. for communicating with a remote server, e.g. a doctor's surgery, and/or for transmitting or transferring data, for example, the tongue composition data of one or more oral muscles of the user. The communication means may comprise a communication element or module or component or device and/or may include a wireless communication or telecommunication means or system or a transmitter or wireless transmitter or receiver or a wireless receiver. Preferably, the communication means is operatively connected to the processor or processing means. More preferably, the apparatus or system is configured or programmed to cause the communication means to transmit, e.g. on or after detection of a muscle composition data, at least some of the data set, for example to a server or remote server. The communication means may be configured or configurable to transmit data (e.g. impedance data, tongue composition data, data relating to the tongue composition, etc.) to an application, e.g. on a or the computing device. The computing device may be remote or local to the apparatus. The computing device may be portable, for example hand-held. The computing device may be non-portable. The application on the computing device may be installed thereon and/or running thereon. The application may be configured to carry out conversion of measured data (e.g. impedance, temperature, EMG signal data) into data relating to the tongue composition (e.g. tongue composition data). The application on the computing device may be configured to transmit received data (e.g. the received data), for example to a or the remote server or a remote storage device. The application on the computing device may be configured to transmit received data (e.g. the received data) via the Cloud, for example to a or the remote server or a remote storage device. The remote server or remote storage device may be arranged or provided for access to the data (e.g. impedance data, tongue composition data, data relating to the tongue composition, etc.) by one or more medical professional, relative, patient/user, etc.

The apparatus or system may further comprise a server, e.g. a remote server, which may comprise a server communication means, e.g. for receiving data from the apparatus and/or for sending data to the apparatus. The server communication means may comprise a communication element or module or component or device and/or may include a wireless communication or telecommunication means or system or a transmitter or wireless transmitter or receiver or a wireless receiver.

Another aspect of the methods and apparatuses described herein provides a computer program element comprising computer readable program code for causing a processor to execute a procedure to implement a method, e.g. a method of measuring tongue composition, or treatment regime comprising providing electrical stimulation to one or more oral muscles, e.g. palate and/or tongue muscles, through the lining of the mouth, for example the oral mucosa, e.g. to increase resting muscle function (e.g. tone, responsiveness, etc.) and/or muscle function (e.g. tone, responsiveness, etc.) during sleep. The computer readable program code may be or comprise firmware, for example which may be updated (e.g. periodically).

Another aspect of the methods and apparatuses described herein provides a method of determining the tongue composition of a person, the method comprising;
a) locating one or more electrodes in the mouth of a person;
b) applying electrical stimulation via the one or more electrodes to one or more muscles of the mouth of the person;
c) using a sensing assembly to determine the tongue composition of the person.

Another aspect of the methods and apparatuses described herein provides a method of providing a stimulation plan, the method comprising;
a) locating one or more electrodes in the mouth of a person;
b) applying electrical stimulation via the one or more electrodes to one or more muscles of the mouth of the person;
c) using a sensing assembly to determine the tongue composition of the person;
d) generating a stimulation plan based on determined tongue composition.

Step d) may comprise automatically generating the stimulation plan based on the determined tongue composition.

Another aspect of the methods and apparatuses described herein provides a method of altering (e.g. dynamically) a stimulation plan, the method comprising;
a) providing a stimulation plan for electrically stimulating one or more muscles of a mouth of a person;
b) locating one or more electrodes in the mouth of the person;
c) applying electrical stimulation via the one or more electrodes to one or more muscles of the mouth of the person;
d) using a sensing assembly to determine the tongue composition of the person;
e) adjusting the stimulation plan according to the determined tongue composition of the person.

Step e) may comprise automatically adjusting the stimulation plan according to the determined tongue composition.

The stimulation plan preferably comprises at least two stimulation sessions to be performed by the mouthpiece on the user, for example whilst the user is in an awake state.

In some examples, using the sensing assembly to determine the tongue composition of the person comprises using one or more impedance sensors to measure the impedance at specific locations on the tongue of the person. In some examples, measuring the impedance comprises measuring the impedance at a single frequency over time. In some examples, measuring the impedance comprises measuring the impedance at multiple frequencies, e.g. over a range of frequencies. In some examples, measuring the impedance comprises measuring the impedance at multiple frequencies over time, for example during a sampling window (e.g. between 0 to 90 seconds or longer). The sampling window may be about 20 seconds, about 30 seconds, about 40 seconds, about 50 seconds, about 60 seconds, about 70 seconds, about 80 second, about 90 seconds, etc.

In some examples the method may further comprise determining the tongue composition of the user using bioelectrical impedance vector analysis.

In some examples the method may further comprise determining the tongue composition of the person using electrical impedance tomography, e.g. to produce a tomogram.

The method may comprise altering a successive stimulation session based on the measured composition of the tongue of the user.

A typical stimulation session may comprise stimulating the mouthpiece whilst in the mouth of the user for a certain period of time, for example less than 20 minutes. A stimulation plan may provide one or two or more stimulation sessions a day for plural days, say one, two or three months (e.g. the stimulation plan may comprise 31 or 62, 62 or 124 or 93 or 186 stimulation sessions or more). The apparatus may be configured to change successive stimulation sessions and/or the method may entail altering successive stimulation sessions depending on the tongue composition data.

A yet further aspect of the methods and apparatuses described herein provides a device for applying an electrical stimulation to one or more muscles of the mouth of the user, the device comprising one or more electrodes for applying electrical stimulation to one or more muscles of the mouth of a user and a sensor to sense, determine or measure the tongue composition of the user.

A still further aspect of the methods and apparatuses described herein provides apparatus for applying an electrical stimulation to the mouth of a user, the apparatus comprising a device as stated above and a controller (or control means, e.g. a control unit), the controller (or control means, e.g. control unit) being operable to alter the electrical stimulation to said one or more tongue muscles based on sensed, determined or measured tongue composition of the user sensed, determined or measured by the sensor.

The apparatus may comprise a memory or memory means to hold data relating to one or both of the electrical stimulation which has been applied to said one or more muscles and/or data relating to the measured or sensed tongue composition of the user. The apparatus may comprise a processor to process data relating to one or both of the electrical stimulation which has been applied to said one or more muscles and/or data relating to the measured, determined or sensed tongue composition of the user. The processor may be operable to control the controller (or control means, e.g. control unit) based on data processed by the processor.

Another aspect of the methods and apparatuses described herein provides a method for measuring the tongue composition of the user, the method comprising:
a) providing an apparatus, e.g. an electrical stimulation device, for training oral muscle function (e.g. tone, responsiveness, etc.), the apparatus comprising a mouthpiece having at least one electrode associated with the mouthpiece, electrical circuitry operatively connected to the electrode, wherein the apparatus is configured to provide, in use, via the at least one electrode electrical stimulation to one or more oral muscles, e.g. tongue muscle and optionally palate muscles, through the lining of the mouth, for example the oral mucosa, e.g. to increase resting muscle function (e.g. tone, responsiveness, etc.) and/or muscle function (e.g. tone, responsiveness, etc.) during sleep, the apparatus further comprising a sensor for measuring tongue composition of the user;
b) locating the mouthpiece in a user's mouth;
c) activating the sensor to collect tongue composition data from the tongue of the user;
d) converting the data into user readable tongue composition data.

A further aspect of the methods and apparatuses described herein provides a method of providing a stimulation plan, the method comprising locating a mouthpiece having one or more electrodes in the mouth of a user; using a sensor on the mouthpiece to determine the tongue composition of the user; generating a stimulation plan based on determined tongue composition. The method may comprise automatically generating the stimulation plan based on the determined tongue composition.

A yet further aspect of the methods and apparatuses described herein provides a method of altering a stimulation plan, the method comprising; providing a stimulation plan for electrically stimulating one or more muscles of a mouth of a user; locating a mouthpiece in the mouth of a user; using a sensor on the mouthpiece to determine the tongue composition of the user; and adjusting the stimulation plan according to the determined tongue composition of the user. The method may comprise automatically adjusting the stimulation plan according to the determined tongue composition of the user.

The method may further comprise providing user-related data, for example, one or more of age, weight, height, BMI. The method may also comprise comparing the determined tongue composition to a desired or expected tongue composition and adjusting the stimulation plan accordingly. The simulation plan may be automatically adjusted.

The stimulation plan preferably comprises one or more stimulation sessions. The electrodes may be controlled according to certain control paradigms during a stimulation session. The control paradigms may relate to current, current type, electrical intensity, frequency, pulse length, pulse duration, stimulation time and so on.

For example, a stimulation session may comprise a stimulation period of 20 minutes, during which a biphasic current is used to stimulate muscles, the biphasic current having a certain frequency, amplitude, pulse duration. A further or subsequent stimulation session may have the same or different characteristics.

Another aspect of the methods and apparatuses described herein provides a method, comprising inserting a mouthpiece into a subject's mouth so as to be contactable with or by the subject's tongue; sensing, using a sensor positioned on the mouthpiece, a sensed signal comprising one or more of: impedance, temperature, and electromyographic signal, from the subject's tongue; determining a composition of the subject's tongue from the sensed signal; for example and applying a therapeutic electrical signal to the patient's tongue based on the sensed signal.

Another aspect of the methods and apparatuses described herein provides a system for applying electrical stimulation to one or more muscles of the mouth to train the muscles of the mouth for combating sleep disordered breathing, the system comprising a mouthpiece for locating in the mouth of a user, the mouthpiece having one or more electrodes; the mouthpiece having one or more sensors configured to measure the tongue composition of a user; a controller (or control means, e.g. a control unit) for controlling the one or more electrodes and the one or more sensors; a processor for providing signals to and receiving signals from the controller (or control means, e.g. control unit); and wherein the processor is operable to cause the controller (or control means, e.g. control unit) to control the electrodes dependent upon signals received from the sensors.

The sensor or one or more sensors may be an impedance or EMG sensor. In an example the sensors may be able to determine a perturbation or attenuation of an electrical stimulation signal transmitted by the electrodes. The or a stimulation signal may propagate vertically (e.g. between a dorsal and sublingual surface of the tongue) or laterally (e.g. across a dorsal surface of the tongue), or both. The sensors may be any of those set out above.

A further aspect of the methods and apparatuses described herein provides a computer program element comprising computer readable program code for causing a processor to execute a procedure to implement the aforementioned method. A yet further aspect of the methods and apparatuses described herein provides the computer program element embodied on a computer readable medium.

A yet further aspect of the methods and apparatuses described herein provides a controller or control system or control unit or control means comprising the aforementioned computer program element or computer readable medium for measuring the tongue composition of a user, for example for controlling the method described above.

Also described is an apparatus for monitoring progress of a stimulation plan and/or to modify a stimulation plan, the apparatus comprises a processor and a microphone, the microphone being operable to record sounds during periods of sleeping, the processor being operable to analyse said sounds and to compare at least one characteristic of a sound to a corresponding characteristics of one or more previously recorded sounds.

The characteristic may be one or more of the volume of the sound, the duration of a sound above a certain threshold volume, the number of times or the total duration the sound level exceeds a certain threshold volume. For example, the microphone may be able to determining the loudness of a snore, the frequency of snoring events, the number of times a snore exceeds a particular threshold volume or the total time that a particular threshold volume is exceeded during a certain period (e.g. during a period of sleep). If a trend in the characteristic is changing over time the processer can amend a stimulation plan in accordance with the change of the characteristic. For example, if the volume of snoring is decreasing, the processor may change the stimulation plan to provide less overall stimulation or vice versa. By linking the processor to the above apparatus a further, different or alternative check can be applied to any modification of the stimulation plan. Moreover, such an apparatus allows for a ‘real world’ correlation between tongue composition and frequency and or volume of snoring, for example.

Within the scope of this application it is expressly intended that the various aspects, examples and alternatives set out in the preceding paragraphs, in the claims and/or in the following description and drawings, and in particular the individual features thereof, may be taken independently or in any combination. That is, all examples and/or features of any example can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the methods and apparatuses described herein, whether or not these are expressly claimed. The applicant reserves the right to change any originally filed claim or file any new claim accordingly, including the right to amend any originally filed claim to depend from and/or incorporate any feature of any other claim although not originally claimed in that manner.

Also described herein are methods and apparatuses including a sensor assembly configured to measure one or more biomarkers, including one or more biomarkers for detection of a co-morbidity of snoring and/or sleep apnoea, such as glucose, blood pressure, etc. For example, any of the methods or apparatuses described herein may include a sensor or sensing assembly configured to use saliva, and/or interstitial content data to identify glucose levels (diabetes), blood pressure surrogate measure, cardiac disease, fat content etc.

A further aspect of the methods and apparatuses described herein provides an apparatus for applying electrical stimulation to one or more muscles of the mouth of a user, the apparatus comprising one or more electrodes for applying electrical stimulation to one or more muscles of the mouth of a user and a sensing assembly configured or configurable to measure one or more biomarkers for detection of a co-morbidity of snoring and/or sleep apnoea. The one or more biomarkers may comprise glucose, blood pressure, etc. In examples, the sensing assembly may be configured or configurable to use saliva and/or interstitial content data, for example to identify glucose levels (diabetes), a blood pressure surrogate measure, cardiac disease, fat content, etc. In examples, the sensing assembly may be configured or configurable to determine or measure, in use, the tongue composition of the tongue of the user.

For the avoidance of doubt, features and/or steps described with respect to one aspect apply equally with respect to any other aspect herein.

### BRIEF DESCRIPTION OF THE DRAWINGS

Examples of the methods and apparatuses described herein will now be described by way of example only with reference to the accompanying drawings in which:

FIG. 1 is a plan view of a part of the apparatus according to an example of the methods and apparatuses described herein;

FIG. 2 is a side elevation of the of FIG. 1;

FIGS. 3A, 3B, and 3C are respectively perspective, plan, and side elevation views of a part of the apparatus according to a second example of the methods and apparatuses described herein;

FIGS. 3D and 3E show views of the apparatus of FIGS. 3A to 3C in use;

FIGS. 4A and 4B is a mouthpiece, and the mouthpiece in use, for use in an apparatus according to a third example of the methods and apparatuses described herein;

FIG. 5 is a schematic of a human mouth showing the palatoglossus and surface of the tongue;

FIG. 6 is a schematic illustrating the extrinsic tongue muscles of a human tongue;

FIG. 7 is a schematic illustrating the intrinsic tongue muscles of a human tongue;

FIG. 8 is another schematic of a human mouth showing the muscles of the palate;

FIG. 9 is a schematic illustrating further muscles of the palate;

FIG. 10 is a flow diagram illustrating how the tongue composition data is recorded and used to inform a future treatment plan according to a further example;

FIG. 11 is a schematic illustration of electricity flowing through muscle and through fat;

FIG. 12 is a plot showing the change of impedance over time;

FIG. 13 are plots of magnitude and phase against frequency as generated using multi-frequency bioelectrical impedance analysis;

FIG. 14 is a schematic illustration of electrical current passing through biological material and a circuit diagram equivalent corresponding to the same;

FIG. 15a-15d are Cole-Cole graphs of the complex vs the real parts of impedance along with their equivalent electrical circuit models;

FIG. 16 is a plot of impedance vs time of a tongue of a user and a plot of an ECG signal vs time of the heart of the user;

FIG. 17 is a flow diagram illustrating a method of generating an impedance model using a Cole-Cole graph; and

FIG. 18 is a schematic illustration of one example of an apparatus as described herein.

### DETAILED DESCRIPTION

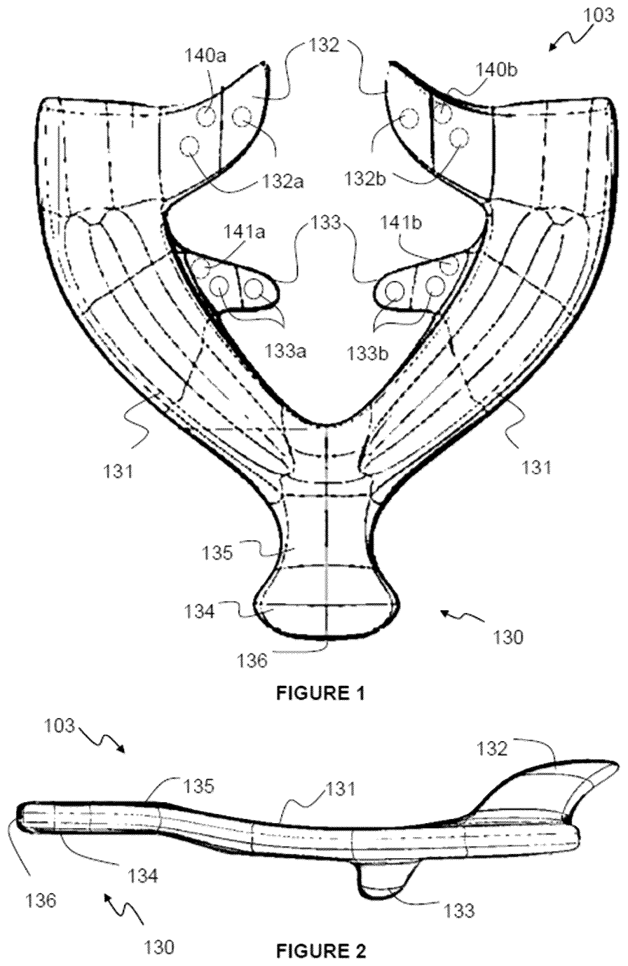

Referring now to FIGS. 1 and 2, there is a shown a component of the apparatus according to a first example of the methods and apparatuses described herein. The FIGS. 1 and 2 show a mouthpiece 103 which includes a gripping base 130 and a pair of curved arms 131 formed integrally with one end of the base 130 to form a horseshoe shape. Each of the arms has first and second contact flanges 132, 133 within are provided electrodes (132a, 132b; 133a, 133b).

The first contact flanges 132 extend inwardly toward one another from the free end of a respective one of the arms 131 and upwardly to form a curved shape for accommodating the dorsal tongue surface 57 (FIG. 9) of a tongue of a user. The second contact flanges 133 extend inwardly toward one another from an intermediate part of a respective one of the arms 131 and downwardly to form a curved shape for accommodating the sublingual tongue surface. As viewed from the side (FIG. 2), the electrode or electrodes 132a on the first contact flanges 132 will face downwardly whereas those electrodes 133a, 133b on the second contact flanges 133 will face upwardly. In this way, with the user's tongue located between the first 132 and second 133 contact flanges the electrodes 132a, 132b; 133a, 133b will apply an electrical field in a vertical direction and will specifically target the genioglossus muscle.

As shown in FIG. 1, the mouthpiece 103 further comprises two sets of sensors (140a, 140b; and 141a, 141b) for measuring the tongue composition of the user.

The first set of sensors (140a, 140b) is located on the first contact flange 132, such that each of the first contact flanges 132 comprises a pair of sensors, 140a or 140b. The second set of sensors 141a, 141b, is located on the second contact flanges 133, such that each of the second contact flanges 133 comprises a pair of sensors, 141a or 141b. However, it is to be understood that at least some of the sensors 140a, 140b or 141a, 141b may not be present, such that only one sensor, or one set of sensors is present, e.g. on the first contact flanges 132, or on the second contact flanges 133.

In alternative examples, the sensors may be located at different locations on the mouthpiece 103, e.g. at an appropriate location on one of the arms. By providing the sensors at different locations or positions on the apparatus, it is possible to measure the composition of the tongue in different regions or parts of the tongue.

The base 130 includes an enlarged end 134 joined to the arms 131 by a necked portion 135. The end surface of the enlarged end 134 includes an electrical connector 136 for connection with a source of power (not shown). The connector 136 may comprise a USB, microUSB, USB-C, Fire Wire®, Thuderbolt®, magnetic connectors or any other suitable type of wired connector. In other examples, the connector is replaced with a wireless connection (e.g. wireless connection means). In some examples, the mouthpiece incorporates a power source, such as a battery.

The mouthpiece 103 also includes electrical circuitry (not shown) connecting the respective series of electrodes 132a, 132b, 133a, 133b at each surface of each flange 132, 133, that is to say each of the upper and lower surfaces of each of the flanges 132, 133. The electrical circuitry (not shown) may also connect the respective sets of sensors 140a, 140b, and 141a, 141b.

Each of these electrode series 132a, 132b; 133a, 133b is electrically isolated from the others by a shielding material, thereby enabling all surrounding muscles to be stimulated simultaneously or in any sequence required. The electrodes 132a, 132b; 133a, 133b cooperate with the outer surface of the flanges 132, 133 with which they are associated to form a substantially contiguous surface. In this example, the mouthpiece 103 is formed of a food grade or a biocompatible grade plastic material, for example made from silicone plastics material. The electrodes 132a, 132b; 133a, 133b in this example are preferably formed of metal, for example gold, silver or copper or composite material or any such alloy with an exposed surface.

In use, the mouthpiece 103 is placed in a patient's mouth and the tongue of the patient is received within the mouthpiece 103 such that the dorsal tongue surface 57 is in contact with the first contact flanges 132 and the sublingual tongue surface is in contact with the second contact flanges 133. It will be appreciated by those skilled in the art that the first flanges 132 will contact a rearward or posterior portion of the dorsal tongue surface 57 and the second flanges 133 will contact a frontward or anterior portion of the sublingual tongue surface. With the patient's mouth closed, the flanges 132, 133 are also able to contact and stimulate adjacent muscles on the other side of the tongue, for example the palate muscles.

During a stimulation session, the mouthpiece 103 enables the muscles to be stimulated, for example on both sides of the tongue simultaneously. It will be appreciated that with this design, the muscles based in and around the tongue may be stimulated, including those in hard and soft palate areas.

Before, during, or after stimulation of the muscles based in and around the tongue, the sensors (140a, 140b; and 141a, 141b) may be used to take measurements to determine the composition of the tongue of the user.

In one example, one or more or all of the sensors (140a, 140b; and 141a, 141b) may be impedance sensors, which measure the bioimpedance of the tongue of the user at different locations. The data may be usable to determine the tongue composition and changes in tongue composition over time. The measurements from the impedance sensors may be used to provide an image (e.g. using electrical impedance tomography) of the tongue composition of the user.

Additionally or alternatively, one or more or all of the sensors (140a, 140b; and 141a, 141b) may be temperature sensors (e.g. a thermistor), which measure the temperature change at different locations of the tongue. This data is usable to be converted into information about the tongue composition of the user.

Additionally or alternatively, one or more or all of the sensors (140a, 140b; and 141a, 141b) may be electromyograph sensors for performing electromyography (EMG). The measurements from the electromyograph sensors may be used to provide an electromyogram, which provides information about the tongue composition of the user.

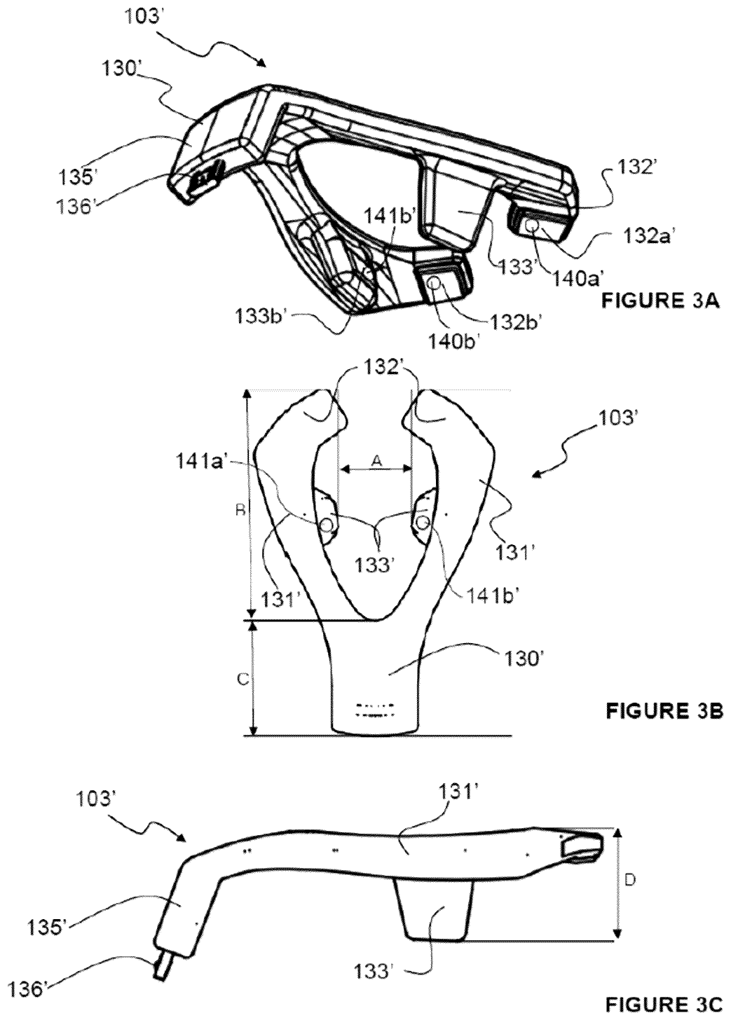

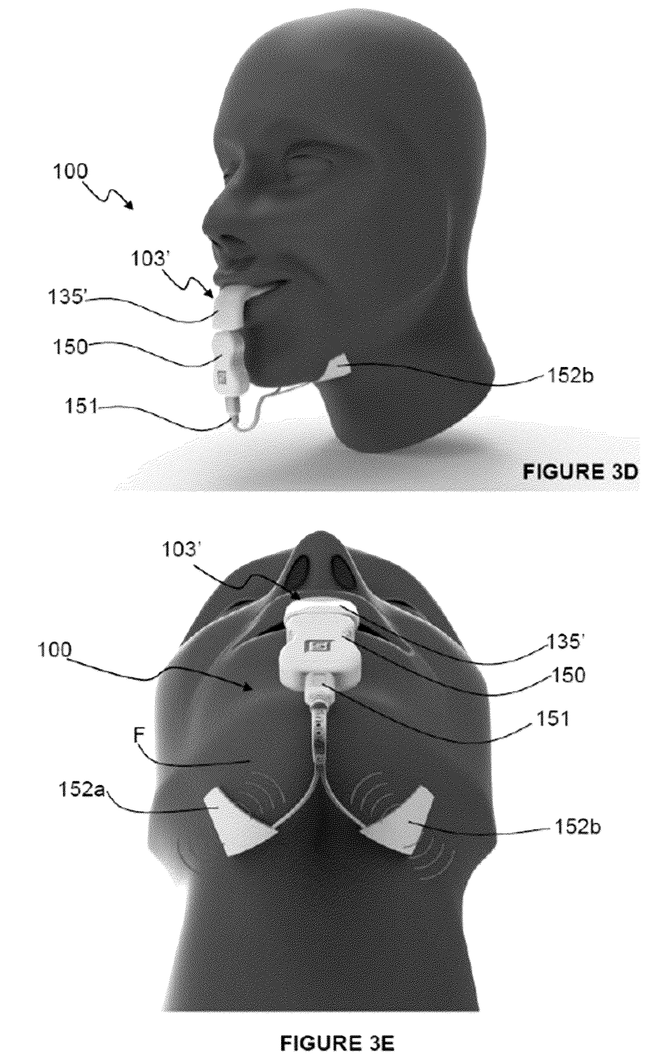

Referring now to FIGS. 3A, 3B, and 3C, there is shown a mouthpiece 103′ according to a second example of the methods and apparatuses described herein. The mouthpiece 103′ is similar to that shown in FIGS. 1 and 2 (and uses the same numerals to indicate the equivalent components but distinguished by a prime (′)) and is shown in FIGS. 3A, 3B, and 3C.

The mouthpiece 103′ is suitable for use in a similar manner to that described for the mouthpiece 103 of FIG. 1. The mouthpiece 103′ is placed in a patient's mouth and the tongue of the patient is received within the mouthpiece 103′ such that the dorsal tongue surface 57 is in contact with the first contact flanges 132′ and the sublingual tongue surface is in contact with the second contact flanges 133′.

Of note, the first contact flanges 132′ extend as a continuation of the longitudinal axis of the arms 131 and from a free end of each arm 131′. The second contact flanges 132′ extend downwardly from a mid-point of each arm 131′. The mouthpiece 103′ has a gripping base 130′ which has an extended and depending portion 135′ which extends over a user's bottom lip (see FIG. 3D).

The terminal portion of the depending portion 130′ has an interface 136′ for engaging with a control and/or power unit (see FIGS. 3D and 3E) which is arranged to provide the power to the electrodes 132a′, 132b′, 133a′, 133b′ of the mouthpiece 103′ and, optionally, to further optional electrodes. It is noted that the electrodes 132a′, 132b′, 133a′, 133b′ of the mouthpiece 103′ are shown as single pads, although they could be multiple pads or contact points. We prefer a single pad as it provides a large surface area. The electrodes 132a′, 132b′, 133a′, 133b′ of the mouthpiece 103′ protrude proud of the adjacent portions of the associated flanges 132′, 133′ to facilitate a good connection with the facing portion of the user's tongue.

In this example, the sensor comprise four single sensors 140a′, 140b′, and 141a′, 141b′. Each flange of the first contact flanges 132′ comprises a single sensor; either 140a′ or 140b. Each flange of the second contact flanges 133′ comprises a single sensor; either 141a′ (not shown in FIG. 3A) or 141b′. The sensors 140a′, 140b′, and 141a′, 141b′ may be located on or under the electrodes 132a′, 132b′, 133a′, 133b′. One or more of the sensors 140a′, 140b′, and 141a′, 141b′ may be absent.

As described for the mouthpiece 103 of FIG. 1, the sensors may comprise or be one or more of impedance sensors, temperature sensors, or EMG sensors.

As shown in FIGS. 3D and 3E, the mouthpiece 103′ interfaces with a controller or control unit 150 via connection 136′. The controller or control unit 150 may also be connected to optional further electrodes 152a, 152b for stimulating muscles in the floor of the user's mouth by attachment to the external surface of the floor of the user's mouth F.

The control unit 150 and sensors 140a′, 140b′, and 141a′, 141b′ may comprise sensing assembly.

In order to train the muscles of the mouth, the control unit 150 is programmed (or a pre-programmed program is selected) and the mouthpiece 103′ and optional electrodes 152a, 152b are connected to the control unit 150 to deliver electrical stimulation to various muscles according to a stimulation plan. Once the program has started, the control unit 150 will energise the electrodes 132a′, 132b′, 133a′, 133b′ (and optionally 152a, 152b) according to the required or desired stimulation plan or profile to apply the electrical stimulation to the muscles.

The control unit 150 may comprise batteries and logic and control circuitry (not shown) to control the application of electric currents to the various electrodes.

The control unit 150 is also programmed or programmable to measure the composition of the tongue of the user. At certain times during delivery of the stimulation plan, or at a convenient time after or before commencement of a stimulation plan, a test mode may be selected or activated. During the test mode the user will use the mouthpiece 103, 103′ to determine the tongue composition of the user.

In some examples wherein one or more of the sensors is an impedance sensor, the apparatus, e.g. the control unit 150, may further comprise an impedance converter (not shown). The impedance converter may comprise an on-board frequency generator (e.g. 12 bit, 1 mega sample per second, MSPS). The impedance converter comprises an analogue to digital converter, an on-board digital signal processor (DSP), and a sine wave generator. The impedance converter is configured to communicate with an Asynchronous Serial Interface Circuit (ASIC) (e.g. using Python® or another programming language). The impedance converter also comprises an interface to read data (e.g. APP, or a personal computer (PC)). The impedance converter also comprises software to analyse data (e.g. Excel®, Matlab®, Python®).

An example of an impedance converter is available from Analog Device Inc. (AD5933). The converter has 2.7 V to 5.5 V, 250 kSPS, 12-bit impedance converter, with an internal temperature sensor and is packaged in a 16-lead SSOP.

In a method to determine the tongue composition of a user, the impedance of the tongue is taken by one or more sensors on the mouthpiece. The data is sent to a controller or microcontroller (e.g. control unit 150), the data is processed by the interface (e.g. an APP, or a PC).

The impedance sensors may be used to evaluate the tongue composition at a single (e.g. a specific) frequency over time (e.g. 50 kHz over 2 minutes). Additionally or alternatively, the impedance sensors may be used to evaluate the tongue composition at multiple frequencies, for example, over a range of frequencies (e.g. between 0 to 100 kHz).

The impedance sensors may be usable to perform bioelectrical impedance vector analysis (BIVA) to determine the composition of the tongue of the user. BIVA is a non-invasive method of measuring human body composition, e.g. the composition of the tongue.

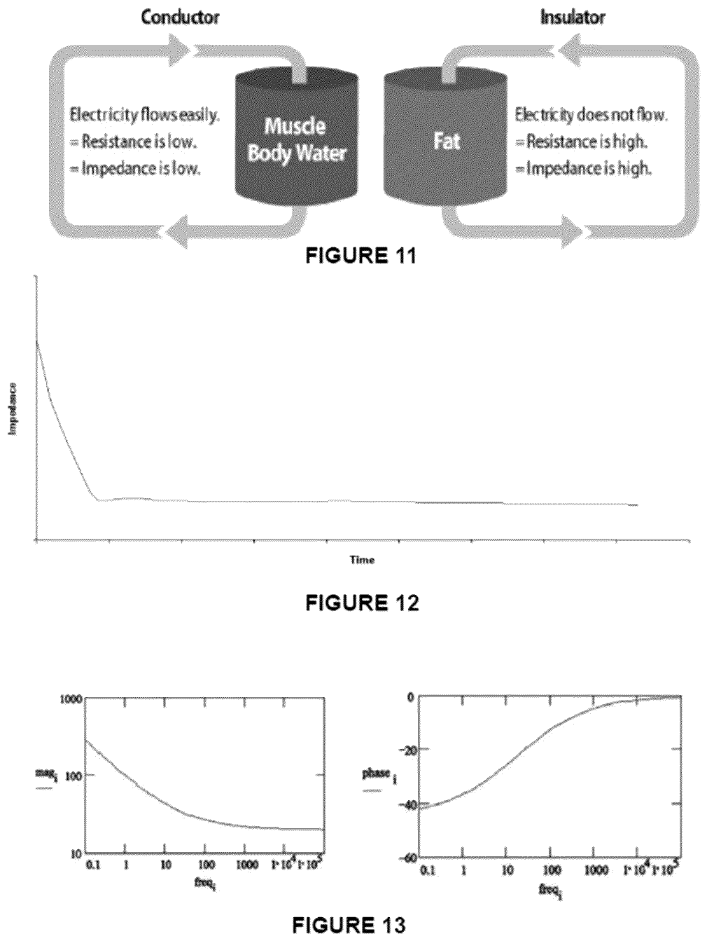

Muscles and other biological materials such as fat are known to exhibit electrically resistive and capacitive properties. For example, as shown in FIG. 11, muscle tends to conduct electricity relatively more readily than does fat. Accordingly, resistance and impedance to electrical flow through muscle is relatively lower than it is through fat.

The impedance is equal to the voltage divided by the current (according to Ohm's law). Accordingly, the voltage and current should be known and/or measured in order to calculate the impedance. In some examples, measurement of the impedance can work with two electrodes. In some examples, this measurement can be applied simultaneously with the pulses generated as part of the electrical stimulation plan.

A constant frequency and constant voltage source may be used for characterization of the load impedance of the tongue. Changes in the composition of the tongue will be observed as changes in the overall impedance. Impedance using a single frequency may be characterized using a single instantaneous measurement (t=0 seconds) or as part of a sampling window (e.g. t=0 to 90 seconds or longer). Advantageously, by capturing time-domain data the capacitive discharge/decay phenomena can be observed over time. This provides additional metrics to this analysis technique, such as the starting impedance, final impedance, decay time and rate of decay. An example of changing impedance over time is shown in FIG. 12.

When multiple frequencies are used instead of a single frequency the voltage source is no longer set to a constant frequency. In this way a spectrum of impedance data may be generated by applying a steadily increasing frequency (for example between 0 to 100 KHz or higher (for example as shown in FIG. 13). Capacitive and resistive elements of the muscle may be interrogated at each individual frequency, allowing for a spectral profile of the equivalent electrical circuit (as explained below).

At lower frequencies the capacitive nature of the model is examined, where the blocking effect increases the impedance. Meanwhile, at higher frequencies the resistive element(s) cause the spectrum to converge at a constant impedance.

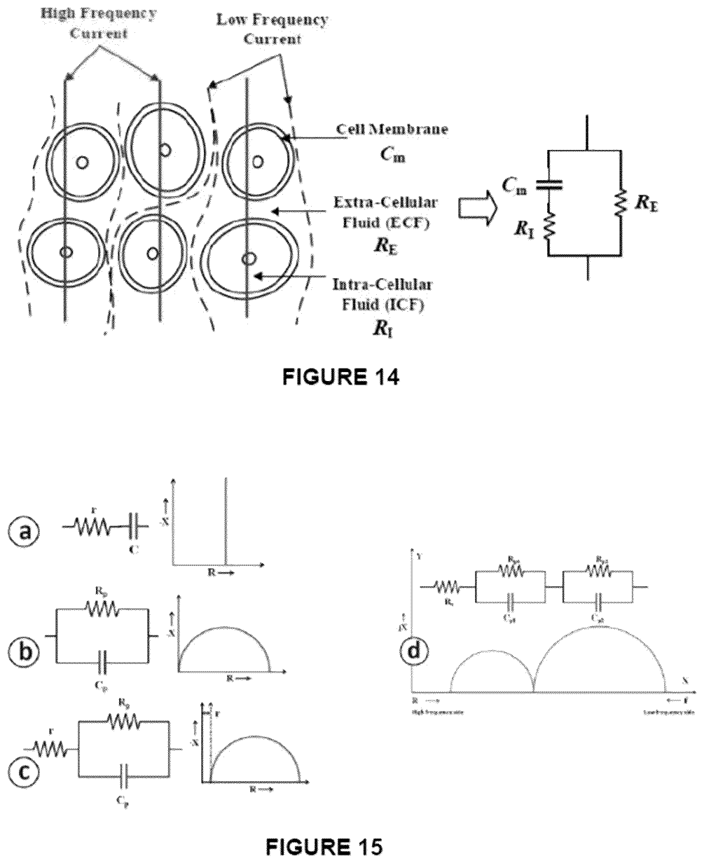

As shown in FIG. 14, at lower frequencies the electrical energy is believed to travel predominately between cell membranes and therefore through the extra-cellular fluid therebetween. At higher frequencies the electrical energy is believed to additionally pass through the cell membranes and hence also through the intra-cellular fluid.

Analysing the circuit model of the biological material shown in FIG. 14 provides the RC circuit contained in that Figure. Allowing the frequency to go to infinity and zero provides the following equations:

$${R}_{∞} = {R}_{I} {R}_{E} / ( {R}_{I} + {R}_{E} )$$

$${R}_{0} = {R}_{E}$$

The equivalent electrical circuit model of the tongue can be identified using Cole-Cole graphs. Such Cole-Cole graphs plot the complex part of impedance (reactance) against the real part of impedance (resistance). Four Cole-Cole graphs are shown in FIGS. 15a-15d, with their associated equivalent electrical circuit models. Use of such Cole-Cole graphs also enables the values of the electrical elements in the equivalent circuits to be ascertained.

Advantageously, once the equivalent electrical circuit model and the relevant circuit values have been identified, the tongue can be emulated using electrical components to verify the results and/or for future prediction and/or simulation. We have found that the component values may change over time. Accordingly, long-term tracking of this model (with sensor measurements and/or therapy) enhances predictive analytics.

The multiple frequency approach can also be used in concert with a time-domain technique (e.g. t=0 to 90 seconds or more). In this way, the spectrum of frequencies can be cycled through a first time, a second time, a third time, etc. Beneficially, this technique provides 3 dimensional data (impedance, frequency and time duration). This allows for a more sophisticated electrical characterization of the tongue composition.

Beneficially, by numerically integrating the area underneath the plot of Cole-Cole graphs it is possible to calculate volumetric data concerning the tongue.

The sensing assembly may be configured or configurable to perform electrical impedance tomography (EIT), for example using impedance data from the, one, some or each impedance sensor (where provided). Electrical impedance tomography (EIT) is a non-invasive type of medical imaging in which the electrical conductivity, permittivity, and impedance of a part of the body is inferred from surface electrode measurements and used to form a tomographic image of that part. The difference in electrical conductivity between the fatty tissue and the muscle mass of the tongue is differentiated by application of small alternating currents at a single frequency or using multiple frequencies (e.g. a range of frequencies). Alternating currents are applied to some or all of the electrodes and the resulting equi-potentials are recorded from the other electrodes. A two-dimension tomogram can be produced by repeating the process for numerous different electrode configurations and applying an image reconstruction algorithm. The data can be used to determine composition of the tongue and/or how compositional change may occur over time.

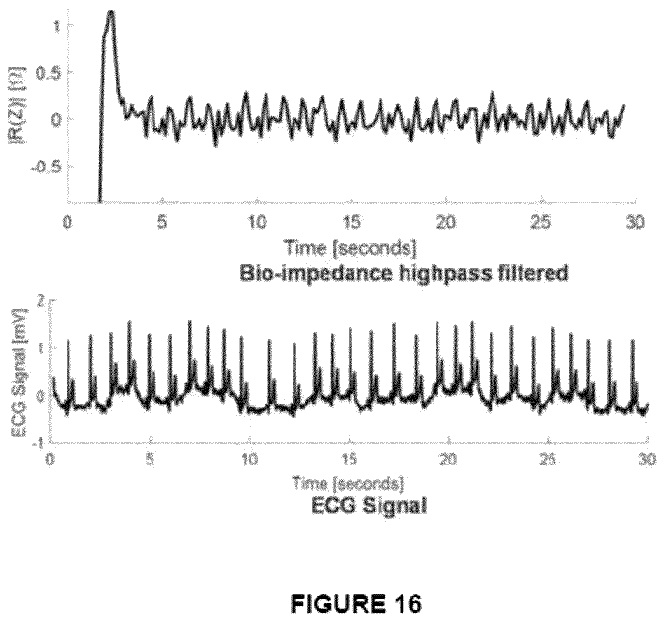

At higher sampling rates and using a high pass filter, we have surprisingly found that it is possible to measure the heart rate of the user from the received sensor readings. FIG. 16 shows a comparison between an ECG signal of a user's heart and a high sample rate of bioimpedance sensor readings of the user's tongue which have been passed through a high pass filter. As can be seen, the heart rate of the user can be clearly distinguished.

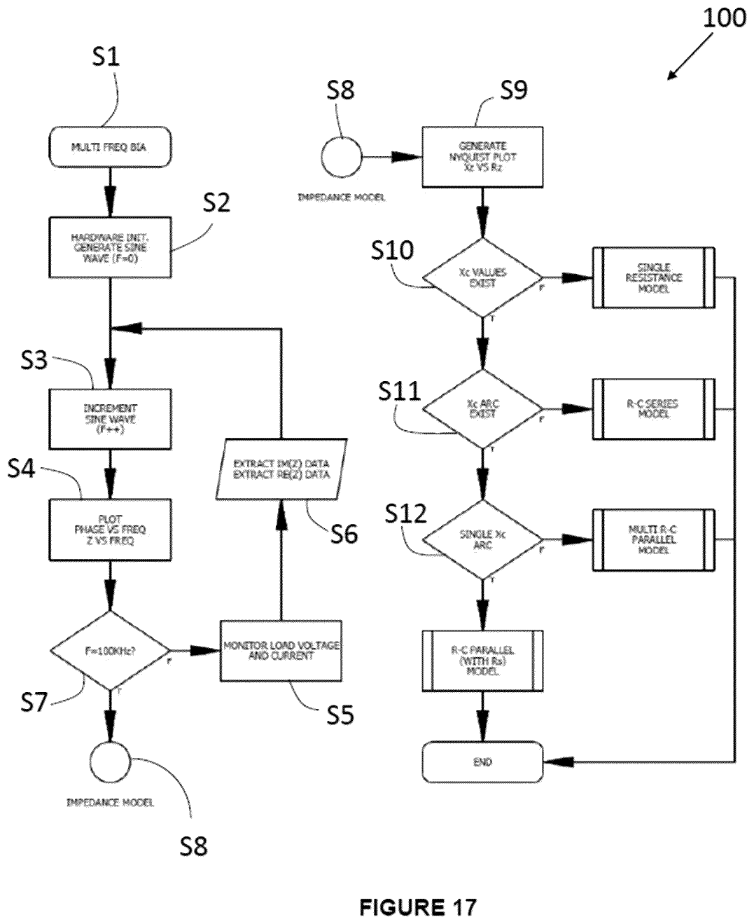

Referring now to FIG. 17, there is shown a method 100 of generating the impedance model using a Cole-Cole graph, as described above. The method 100 comprises a first step S1 of undertaking a multi-frequency bioelectrical impedance analysis of the tongue of a user, as described in greater detail above. This first step S1 comprise a first sub-step S2 of generating a sine wave at a first frequency (for example at frequency 0 Hz). In a second sub-step S3 the sine wave frequency is incrementally increased by a predetermined amount. The sensor reading results obtained at this incrementally increased frequency are plotted as phase vs frequency and Z vs frequency graphs, in a third sub-step S4. The load voltage and current are monitored in fourth sub-step S5. The real and complex components of the impedance at this incrementally increased frequency are extracted in fifth sub-step S6. Sub-steps S3 through S6 are repeated with an incremental increase to the applied frequency until the frequency reaches a predetermined limit (e.g. 100 kHz) at sub-step S7. The results of the multi-frequency bioelectrical impedance analysis are then used to generate an impedance model in step S8. Firstly, an Nyquist plot of the complex vs the real components of the impedance is generated in sub-step S9. In sub-step S10 it is determined whether any Xc (capacitance) value is present. If not, then a single resistance model is generated. If an Xc value is present then in sub-step S11 it is determined whether any Xc arc exists. If no Xc arc is present than an RC series model (for example as shown in FIG. 15a) is generated. If an Xc arc is present then in sub-step S12 it is determined whether a single Xc arc is present. If more than one Xc arc is present than a multi RC parallel model is generated (for example as shown in FIG. 15d). If only one Xc arc is present then a single RC parallel model is generated (for example as shown in FIG. 15b).

In some examples wherein one or more of the sensors is an impedance sensor, the apparatus may be usable to perform electrical impedance tomography (EIT). Electrical impedance tomography (EIT) is a non-invasive type of medical imaging in which the electrical conductivity, permittivity, and impedance of a part of the body is inferred from surface electrode measurements and used to form a tomographic image of that part. The difference in electrical conductivity between the fat content and the muscle mass of the tongue is differentiated by application of small alternating currents at a single frequency or using multiple frequencies.

In use, the mouthpiece (e.g. 103′) is positioned in the mouth of the user. The user's tongue is contacted with the impedance sensors (e.g. 140a′, 140b′, and 141a′, 141b′ of 103′). The control unit (e.g. 150) supplies small alternating currents to one or some or all of the sensors (e.g. 140a′). The resulting equi-potentials are then recorded by the remaining sensors (e.g. 140b′, 141a′, 141b′). This process is repeated for different sensor configurations. The data is then used to produce an image (e.g. a two-dimensional tomogram) of the tongue composition of the user.

In some examples wherein one or more of the sensors is a temperature sensor, the one or more temperature sensors is configured to measure the temperature of specific locations of the tongue. The temperature sensor may comprise or be a thermistor. It is known that vasodilation occurs naturally in body in response to triggers such as low oxygen levels, a decrease in available nutrients, and increases in temperature. When thermoreceptors pick up on a higher amount of warmth in an environment relative to cold, vasodilation will occur. This directs a higher flow of blood toward the skin to dissipate any excess warmth felt. The opposite is true for vasoconstriction. Without wishing to be bound by any particular theory, it is believed that electrical stimulation of oral muscles during a stimulation plan causes the muscle mass to increase, which causes a change in the muscle fibre composition. This has an impact on the perfusion of one or more tongue muscle, and on vasodilatation or vasoconstriction. Therefore, the structure of muscle mass in comparison to fat content generates a different temperature, which is sensed by the sensors of the methods and apparatuses described herein.

In some examples wherein one or more of the sensors is an EMG (electromyograph) sensor, the one or more EMG sensors is configured to record EMG signals from one or more locations on the tongue. EMG signals are recorded by detecting the potential generated by a muscle during contraction.

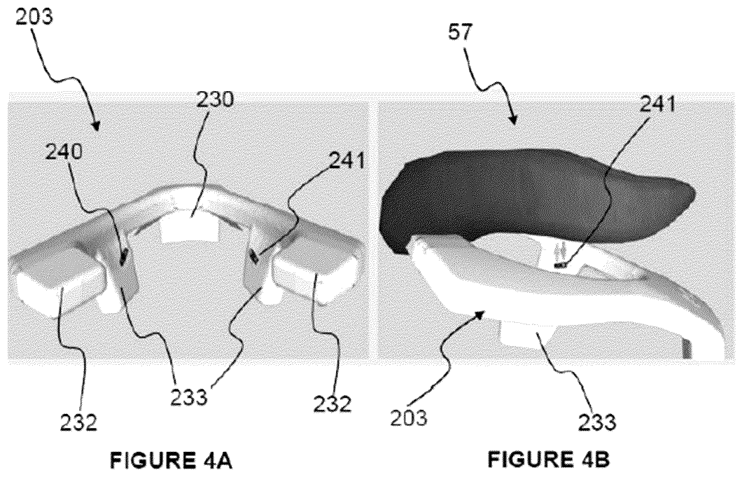

Referring now to FIG. 4A, there is shown a mouthpiece 203 according to a third example of the methods and apparatuses described herein. The mouthpiece 203 is similar to that shown in FIGS. 1, 2, and 3A to 3C and has many of the same features which function in a like manner to that previously described. The mouthpiece 203 comprises a gripping base 230, a first set of contact flanges 232, a second set of contact flanges 233, a first set of electrodes (not shown), and a second set of electrodes (not shown). The first set of electrodes is located on the first set of contact flanges 232. The second set of electrodes is located on the second set of contact flanges 233 in a like manner to that described for the mouthpiece 103′ of FIG. 3A.

In this example, the mouthpiece 203 comprises two sensors 240, 241 for use in determining the composition of the tongue. The sensors 240, 241 are located on the second set of contact flanges 233 adjacent the second set of electrodes (not shown) respectively although they could be mounted in another part of the mouthpiece 203, for example on further flanges (not shown), the first set of contact flanges 232 or the arms. It is preferable to collate the sensors 240, 241, on the flanges (e.g. the second set of contact flanges 233) for fabrication and ease-of-use.

Referring also to FIG. 4B, there is shown the mouthpiece 203 in use located adjacent the tongue of the user. The dorsal surface of the tongue 57 is labelled. It is shown that the sensors 240, 241 contact the underside of the tongue. By providing the sensors 240, 241 at different positions on the mouthpiece 203, it is possible to measure the composition of the tongue in different regions or parts of the tongue.

Each or both sensors 240, 241 in this example is one of an impedance sensor, a temperature sensor, or an EMG sensor.

The mouthpiece 203 may be connected to a control unit (not shown), which is configured to determine the composition of the tongue of the user. Advantageously, the sensors 240, 241 may be integrated into a system for providing feedback to the user on improvements to the tongue composition.

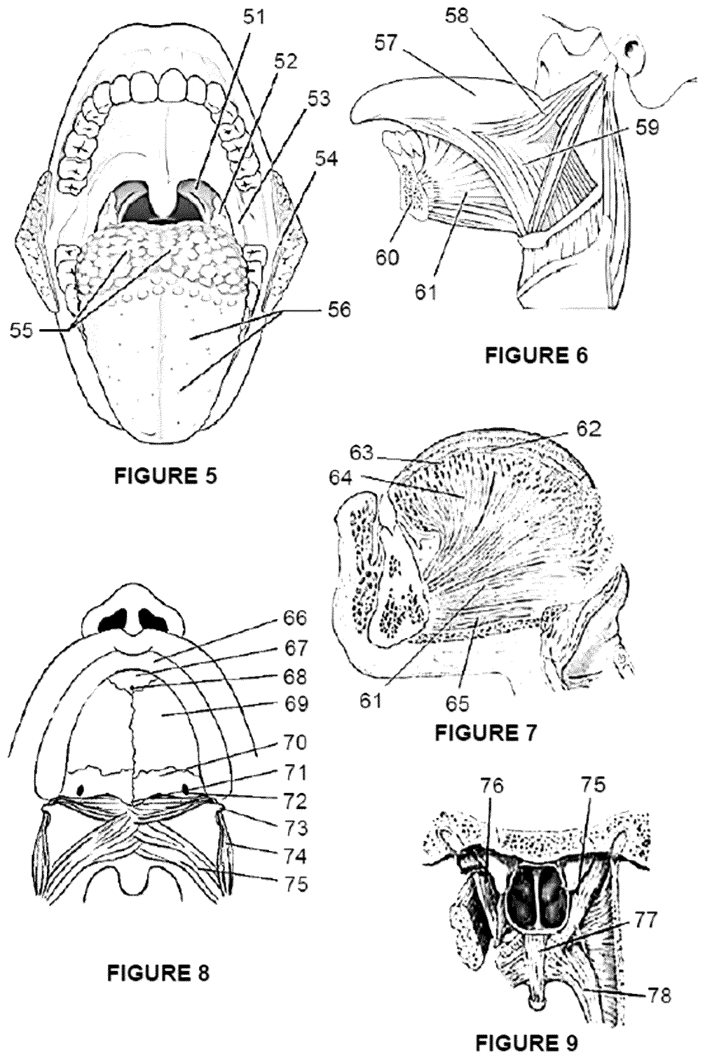

Turning now to FIGS. 5 to 9, there is shown various tongue and palate muscles. Features of the mouth shown in FIGS. 5 to 8 illustrate more clearly the tongue muscles, wherein there is shown the pharyngopalatine arch 51, palatine tonsil 52, palatoglossus 53, buccinator 54, valate papillae 55, fungiform papillae 56, dorsal tongue surface 57, styloglossus 58, hyoglossus 59, mandible bone 60, genioglossus 61, longitudinal, transverse and vertical intrinsic muscles 62, 63, 64 and geniohyoid 65.

It is well established that the tone of the genioglossus muscle 61 most affects the collapsibility of the tongue as it is the biggest of the extrinsic muscle and responsible for pulling the tongue forward and increasing the airway opening in the throat. The tone of intrinsic surface muscles, such as the longitudinal and transverse intrinsic muscles 62, 63, also contribute to the reduction of the collapsibility of the airway. It is also known that patients with a higher quantity of fat content, or a higher ratio of fat content to muscle mass in their tongue, have a higher propensity for the tongue collapsing into the airway.

Features of the mouth shown in FIGS. 8 and 9 illustrate more clearly the palate muscles, wherein there is shown the dental arch 66, premaxilla 67, incisive foramen 68, palatine process of maxilla 69, palatine bone 70, posterior nasal spine 71, palatine foramen 72, hamulus 73, tensor palatini muscle 74, levator veli palatini muscle 75, tensor veli palatini muscle 76, uvular muscle 77 and palatopharyngeus muscle 78.

To a varying degree, the constrictor and dilator muscles of the palate also contribute to snoring and sleep apnoea. The aim of the treatment is to dilate the throat, hence electrical stimulation is directed at the dilatory palate muscles in the midline, such as the uvular muscle 77, the levator veli palatini muscle 75 and the palatopharyngeus muscle 78.

In use, the mouthpiece 103, 103′, 203 is applied to the dorsal tongue surface 57 and/or the sublingual surface and current, for example biphasic currents are applied, each of which may be configured with a first set of parameters including intensity, frequency and pulse duration. The parameters are selected to provide maximal contraction of these muscles in the user and the treatment is carried out for a period of 20 minutes.

The intensity, frequency and pulse duration may then be adjusted and the mouthpiece 103, 103′, 203 is applied to the underside of the tongue and/or the dorsal surface 57. The two currents, for example the two biphasic currents, now having a second set of parameters, are applied and transmitted trans mucosally to stimulate the genioglossus muscle 61. The second set of parameters are selected to provide maximal contraction of the user's genioglossus muscle 61 and the treatment is carried out for a period of, say, up to 3 hours, for example 20 to 30 minutes.

The application of currents, e.g. biphasic currents, according to the parameters described above stimulate the aforementioned skeletal muscles. It is also believed that the application of this biphasic current to these skeletal muscles creates a further, sensory function, such as a vibratory sensation. Whilst not wishing to be bound by any theory, it is believed that this electrical and vibratory stimulation of the nerves provides feed back to the brain which further enhances the improvement in muscle function (e.g. tone, responsiveness, etc.). Specifically, it is believed that the effectiveness of this treatment is enhanced by multisensory integration within the nervous system.

By way of example, a treatment regime could involve a say six-week induction period during which each of the aforementioned muscle groups are stimulated for a period of 10 to 30 minutes, twice daily. The treatment regime, which is designed to build or improve muscle function (e.g. tone, responsiveness, etc.), could then be followed by an ongoing maintenance regime involving 10 to 20 minute sessions once per day.

The apparatus comprising the mouthpiece 103, 103′, 203 may be operable to adjust the current amplitude of a first current, e.g. first biphasic or monophasic current, from 0 to 100 mA. The apparatus may be operable to adjust the current amplitude of, for example, a second biphasic current from 0 to 100 mA. The apparatus may be operable to adjust the duration of the period during which the first current, e.g. biphasic current is supplied from 1 to 30 minutes. The apparatus may be operable to adjust the duration of the period during which the second current e.g. biphasic or monophasic current is supplied from 1 to 30 minutes.

A USB port or other interface may be provided and configured to enable the mouthpiece 103, 103′, 203 to be connected to a personal computer (not shown) to program one or more characteristics of the first and second currents, e.g. biphasic or monophasic currents, independently. In an example, the frequency of the first current, e.g. biphasic current, is set at a value between say 1 and 150 Hz, for example between 2 and 50 Hz, the second current, e.g. second monophasic current, is set at a value between 3 and 120 Hz and the pulse duration of each current, e.g. biphasic or monophasic current, may be set at a value between 200 and 700 μs. The personal computer, tablet, smartphone or other hand-held computing device (not shown) may also incorporate control software operable to override any, say, dials or buttons or other user interface on the control body. The software may be programmed to apply currents, e.g. biphasic or monophasic currents, having predetermined characteristics independent from one another, such as amplitudes, frequencies and pulse durations and for a predetermined period of time.

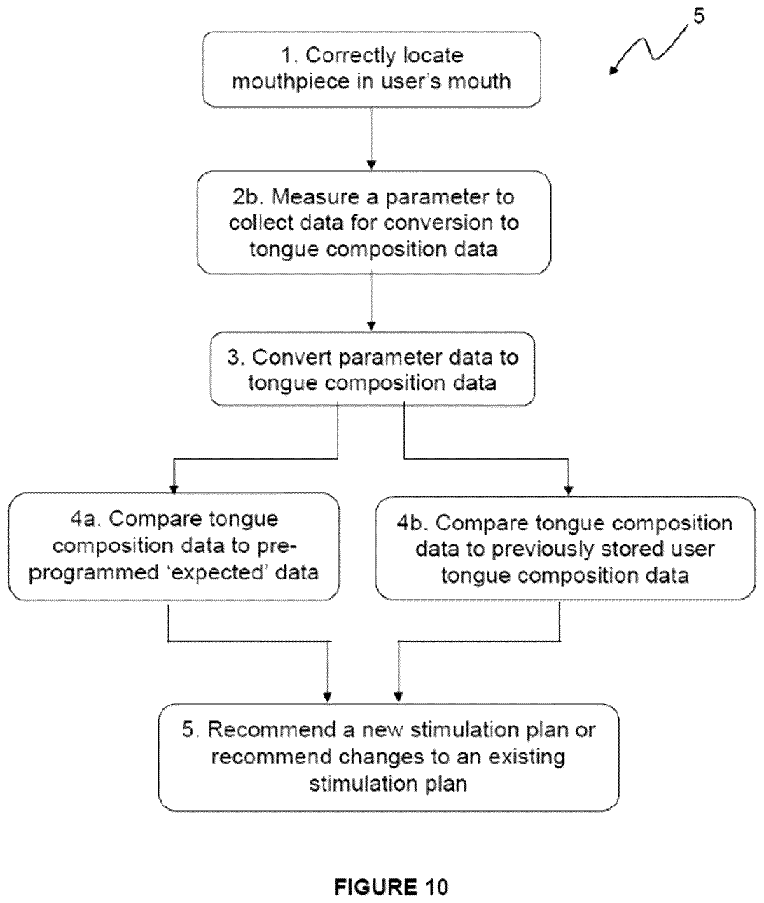

Additionally or alternatively, the software may be programmed to run a measurement of the tongue composition of the user, e.g. using a protocol as described in FIG. 10 below. It is further envisaged that the mouthpiece 103, 103′, 203 could incorporate a memory on which is stored such predetermined characteristics, which may be modified by connecting a personal computer or so on (not shown) to the mouthpiece 103, 103′, 203 via the USB port or other interface. In such examples, the dials may be omitted or configured to adjust the aforementioned characteristics from their pre-programmed values. In some examples, it is envisaged that more or less functionality is provided by manual dials, buttons and the like.

Referring now to FIG. 10, there is shown a flow diagram 10 illustrating how tongue composition data may be recorded using the apparatus (e.g. apparatus comprising the mouthpieces 103, 103′, 203 shown in FIGS. 1, 3A, 4A) and system of the methods and apparatuses described herein, and how the data may be used to inform a future stimulation plan, according to a further example of the methods and apparatuses described herein. The flow diagram comprises the following steps:

### Step 1: Correctly Locate Mouthpiece in User's Mouth.

Before, after, during or instead of a stimulation session, the user may locate the mouthpiece, e.g. mouthpiece 103, 103′, 203 inside their mouth in the same fashion as is described for use during a stimulation session.

### Step 2: Measure a Parameter to Collect Data for Conversion to Tongue Composition Data.

Depending on which sensor is employed, the user may select the program. For example, the program may be one of the following:
Measure tongue composition using bioimpedance analysis;
Measure tongue composition using temperature analysis;
Measure tongue composition using electromyography.

This step may be performed, for example, using the control unit 150 and/or a user interface (for example a computer software program operably connected to the controller 150). Advantageously, the user does not need to actively participate during this measurement.

The control unit 150 or the user interface may issue instructions for the user to maintain their tongue in a specific position until appropriate measurements have been taken from the sensors of the mouthpiece, e.g. 103, 103′, 203.

### Step 3: Convert Data to Tongue Composition Data.

The system may comprise a memory or memory means on which is stored a database for the conversion of data from the sensors of mouthpiece into tongue composition data, and a processor, operably connected to said sensors of the mouthpiece and to the memory or memory means. In Step 3, the system is configured to determine the tongue composition of the user, by comparing the data from the sensors with the data within the database of the memory or memory means, using the processor or processing means. In this way, the user is provided with a user readable or user interpretable value or output corresponding to the tongue composition.

### Step 4a: Compare Tongue Composition Data to Pre-Programmed ‘Healthy’ Data

Optionally, the system may perform Step 4a, which enables a comparison between pre-programmed ‘expected’ data, i.e. the expected tongue composition data from a user who has undergone the stimulation plan, and the user's tongue composition data from Step 3a or Step 3b.

### Step 4b: Compare Tongue Composition Data to Previously Stored User Tongue Composition Data

Optionally, the system may perform Step 4b, in which tongue composition data previously collected from the user, e.g. at an earlier date or point in time or an earlier stage in a stimulation plan, is compared with the data collected in Step 2a, or Step 2b, and converted in Step 3.

For example, comparison of previously stored user tongue composition data may comprise comparison of measured impedance data, e.g. comparison of a bioelectrical impedance spectra. Additionally or alternatively, comparison of previously stored user tongue composition data may comprise comparison of one or more electrical impedance tomograms.

### Step 5: Recommend a New Stimulation Plan or Recommend Changes to an Existing Stimulation Plan

Optionally, the system may be programmed or programmable to be able to make a recommendation of a new stimulation plan, e.g. for a new user, or for a user who has reached the end of their stimulation plan. Alternatively, the system may be programmed or programmable to be able to make a recommendation of changes to an existing stimulation plan.

If the mouthpiece 103, 103′, 203 is used before any stimulation sessions it may be used to determine or establish a base point from which stimulation can begin. For example, when the user uses the mouthpiece 103, 103′, 203 for the first time, ‘Test Mode’ may be selected (or may automatically select and the user is instructed to determine tongue composition data. The mouthpiece 103, 103′, 203 can measure the relevant parameters of the tongue composition and, via an interface (e.g. a computer software program held on a computing device operable connected to the system, for example via controller 150), compare those parameters with a database of such parameters to determine the risk of SDB, for example the likely risk of snoring versus the likely risk of OSA. Other data, for example one or more of age, sex, weight, height, BMI or other indicators may also be input to help with the risk determination. Once a risk profile has been established, a stimulation plan will be developed specific to the inputs.

The user will then use the mouthpiece 103, 103′, 203 according to the stimulation plan, with the requisite stimulation sessions. For example, the stimulation sessions may comprise a daily routine of 20-minute sessions for two weeks, with energisation of the electrodes according to stimulation parameters (current, pulse width, pulse duration, frequency, amplitude) preferably during an awake state.

At the end of the stimulation plan the system may automatically activate (or a user may activate) a Test Mode to determine the efficacy of the stimulation plan by measuring tongue composition. A comparison may then be completed to assess progress (i.e. changes to tongue composition) against expected or predicted tongue composition. A subsequent stimulation plan may then be developed according to the comparison. The subsequent stimulation plan may be the same or different as the first stimulation plan depending upon the results of the comparison.

The changes to an existing stimulation plan may include changes to parameters of the stimulation sessions of a stimulation plan including the intensity, frequency, and pulse duration of electric current being supplied to one or more electrodes of the apparatus, and/or the length of one or more of the stimulation sessions. In this way, the user is able to use the system of the methods and apparatuses described herein to inform them of future stimulation plans, or changes that should be made to existing or ongoing stimulation plans.

It will be appreciated by those skilled in the art that several variations to the aforementioned examples are envisaged without departing from the scope of the methods and apparatuses described herein. For example, the mouthpiece 103, 103′, 203 may take any suitable form, but is preferably designed to enable the electrical stimulation to be applied to the appropriate muscles as described above. The output of the control body may be varied by changing dials on the body itself or it may be altered by interfacing the control unit (not shown) with, for example software, such as an APP held on a mobile device, such as a personal computer, smart phone or tablet. The software may be programmed to apply desired or required currents, for example biphasic currents, having predetermined characteristics (current, duration, frequency) independent from one another, such as amplitudes, frequencies and pulse durations and for a predetermined period of time. It is further envisaged that the apparatus could incorporate a memory on which is stored such predetermined characteristics, which may be modified by connecting a personal computer (not shown) to the apparatus via a USB port or other interface connection. Other interface connections include wired and wireless connections, for example Bluetooth®, Wi-Fi and so on. Other sensors may be deployed which allow for the measurement of the tongue composition of the user.

As will be appreciated, the mouthpiece 103, 103′, 203 can be used as a diagnostic tool to determine the likelihood of SDB (e.g. snoring or OSA) by using the mouthpiece 103, 103′ prior to using the mouthpiece for a stimulation session.

The methods and apparatuses described herein may include any appropriate sensor assembly. As discussed above, a sensor assembly may include a sensor and processing (including a dedicated processor, or may be processed using a portion of the system processor(s)) to detect, filter, amplify and analyse the signal(s) received.

In some variations the sensor assembly may be configured for impedance measuring. An impedance sensing system may be configured to determine and/or monitor the impedance across the tongue before, during and/or after a treatment session or treatment plan. The impedance sensing system may include electrodes for use in the determination of impedance. In an embodiment the electrodes of the mouthpiece may be used to monitor or determine impedance of the tongue. For example, the same electrodes used to apply energy to treat the tongue may be used to sense impedance. In some cases, the impedance measurements may be made using other devices or electrodes, which may be dedicated impedance electrodes.

In one example of an apparatus described herein, which may include an impedance sensing system configured to detect the composition of a muscle in the oral cavity (e.g., the tongue), the apparatus may be include a sensing assembly configured as a high-precision impedance converter. In this example, the sensing assembly may include a frequency generator (e.g., an on-board frequency generator) which may include, e.g., a 12 bit, 1 MSPS (mega sample per second), analog-to-digital converter (ADC). The sensing assembly may also include a digital signal processor, such as an on-board Digital Signal Processor (DSP). The assembly may include a sine-wave generator (which may be part of the frequency generator or functionally connected to it). Any of the sensing assemblies described herein may also include communication circuitry, and/or communication protocols that allow communication between the sensing assembly and one or processors of the rest of the apparatus, such as with an MCU (e.g., I2C). For example, a sensing assembly may include software to communicate with an ASIC (Asynchronous Serial Interface Circuit), using, e.g., python or any other programming language. Any of these sensing assemblies may also include an interface to read data (e.g., APP, PC, remote, etc.) and/or software to analyse data (e.g., Excel, Matlab, R, Python etc). In one example the apparatuses described herein may include a sensing assembly configured to sense impedance that uses an AD5933, a 2.7 V to 5.5 V, 250 kSPS, 12-bit impedance converter, with an internal temperature sensor and is packaged in a 16-lead SSOP (Analog Devices). For example, a sensing assembly may have a programmable output peak-to-peak excitation voltage to a maximum frequency of 100 kHz, an output excitation voltage of between about 0.1 Vp-p, 0.2 Vp-p, 1 Vp-p, 2 Vp-p, etc, an output frequency range of between (0 to 1 kHz), (20 Hz-100 kHz), (1 kHz-100 kHz), a programmable frequency sweep capability with serial interface, a frequency resolution of 27 bit (<0.1 Hz), and/or an impedance measurement range from 1 kΩ to 10 MΩ (e.g., from 100Ω to 1 kΩ, with adaptation for low impedance with extra buffer circuitry). In some examples the Vdd may be from about 2.2 v to 5.7 v, and the current range may be between about (±0.002 mA to ±2 mA) (±0.2 mA to ±20 mA); the sensing assembly may fix the voltage or the current delivered. For example, the impedance sensing may apply galvanostatic excitation (constant flow of current) or potentiostatic excitation (e.g., keep the voltage between a working electrode and a reference electrode at a constant value).

In general terms, an apparatus including a bioimpedance sensing assembly may include an analysis of one or more types or levels. For example, bioimpedance analysis may be single-frequency bioelectrical impedance analysis (SF-BIA). In this configuration, the bioimpedance of one or more tongue muscle may be evaluated at a specific frequency over a range of time (e.g., at about 50 kHz over 2 min). Alternatively or additionally, the apparatus may perform multi-frequencies bioelectrical impedance analysis (MF-BIA), in which the bioimpedance of one or more tongue muscle is evaluated at different frequencies (e.g., within a range, such as from 0 to 100 kHz, e.g., 0-120 kHz, 0-150 kHz, 0-500 kHz, etc.). Alternatively or additionally, the apparatus may perform bioelectrical impedance vector analysis (BIVA), to evaluate bioimpedance at a single frequency, processing the data as a vector that identifies the status of one or more muscles analysed. Alternatively or additionally, the apparatus may be configured to perform bioelectrical impedance spectroscopy (BIS), evaluating bioimpedance using mathematical modelling and mixture equations (e.g., Cole-Cole plot and Hanai formula) to generate a relationship between R (resistance of the tissue) and fluid compartments, or to predict extracellular water (ECW), intracellular water (ICW), and/or total body water (TBW). In some variations, ratio of bioimpedance (RBI) might be used, which may be calculated as a ratio of impedance at a first frequency (e.g., 50 kHz) divided by the impedance at a second frequency (e.g., 500 kHz).

The apparatuses and methods using them may perform one or more of these analysis types or levels, and may switch between them, typically without modification of the apparatus. The analysis may be controlled using software, firmware, hardware, or a combination of these. For example, any of these apparatuses may include non-volatile memory storing instructions for performing any of these analysis types, including controlling the sensor assembly (e.g., for examples configured for measuring a bioimpedance indicator, the system may control the range of frequencies used by the device to evaluate the bioimpedance).

As mentioned above, any of these apparatuses and methods may also or alternatively determine electrical impedance tomography of the tongue or other tissue (e.g., muscles). This may include generating imaging data and/or extracting information from imaging data that may be stored, transmitted, processed, and/or used to control the operation of the apparatus (e.g., treatment session or treatment plan).

For example, an apparatus configured to electrical impedance tomography (EIT), may be configured to determine or monitor the impedance across the tongue before, during and/or after a treatment session or treatment plan. The sensing assembly may include electrodes for use in determining impedance, as described above. For example, the same electrodes of the mouthpiece used to apply therapy to the tongue may be used to monitor or determine impedance of the tongue; alternatively, other (or dedicated) electrodes may be used.

In EIT the electrical conductivity, permittivity, and impedance of a part of the body is inferred from surface electrode measurements and used to form a tomographic image of that part. Electrical conductivity typically varies considerably among various biological tissues (absolute EIT) or the movement of fluids and gases within tissues (difference EIT). When the apparatus is configured to employ EIT, it may apply small alternating currents at a single frequency, or alternatively the apparatus may use multiple frequencies to better differentiate between normal and suspected abnormal tissue within the same organ (multifrequency-EIT or electrical impedance spectroscopy). In some examples, conducting surface electrodes are connected to the oral cavity, e.g., around the tongue or other body part being examined, and small alternating currents may be applied to some or all of the surrounding electrodes. The resulting equi-potentials may be recorded from the other electrodes. This process may then be repeated for numerous different electrode configurations and may result in a two-dimensional tomogram and the apparatus may apply image reconstruction algorithms. In contrast to linear x-rays used in Computed Tomography, electric currents travel three dimensionally along the path of least resistivity.

As mentioned, any of these apparatuses may be configured to detect temperature or temperature response (e.g., response of the surrounding tissue to an induced change in temperature). Thus, in examples in which the apparatus include a sensor assembly that is configured to detect temperature, the sensing assembly may include one or more sensors suitable for use in recording temperature of one or more muscles. For example, the sensing assembly may include one or more thermal sensors (and in some variations a thermal source, such as a resistive heat source, to induce a known change in temperature). In some examples, the sensing assembly may comprise one or more thermistors. During contraction of one or more tongue muscles, a change in temperature and heat produced by those muscles may be observed. One or more sensors comprising one or more temperature sensors, e.g. one or more thermistors, may be configured to detect changes in the temperature produced by the tongue muscles of the user.

Vasodilation may occur naturally in body in response to triggers such as low oxygen levels, a decrease in available nutrients, and increases in temperature. When thermoreceptors pick up on a higher amount of warmth in an environment relative to cold, vasodilation will occur. This directs a higher flow of blood toward the skin to dissipate any excess warmth felt. The opposite may occur for vasoconstriction. There is a change in the muscle fiber composition that may have an impact on the perfusion of one or more tongue muscle and so on vasodilatation or vasoconstriction, and so on the temperature of the muscle structure. Thus, these apparatuses may infer muscle composition based on temperature.

As discussed above, any of these apparatuses may also or alternatively be configured to detect electromyogram (EMG). For example, a sensing assembly may include one or more electrodes suitable for use in recording an EMG signal of one or more muscles. The sensing assembly may include one or more electrodes adapted to detect EMG; these one or more electrodes can be used to detect the potential generated by the muscle during a contraction. The apparatuses and methods described herein may be configured to apply and detect a response to an electrical stimulation device, which is able to measure the muscle composition in terms of electromyograph signal of one or more oral muscles of the user such that the user and/or another (for example a healthcare professional) is able to monitor the progress of the user's stimulation plan.

In any of the methods and apparatuses described herein, the data generated for the muscle composition of one or more oral muscles of the user may be used to modify the treatment plan in response to measured data. For example, a processor can determine progress or change in muscle composition compared to an expected or predicted value and alter the stimulation plan accordingly. Additionally or alternatively, the change in composition detected by the sensor assembly might be communicated to the user (or another) for example visually, aurally or otherwise. In some cases, the detected muscle composition may be used to set the value (e.g., initial value or ongoing value) of the applied treatment session and/or treatment plan.

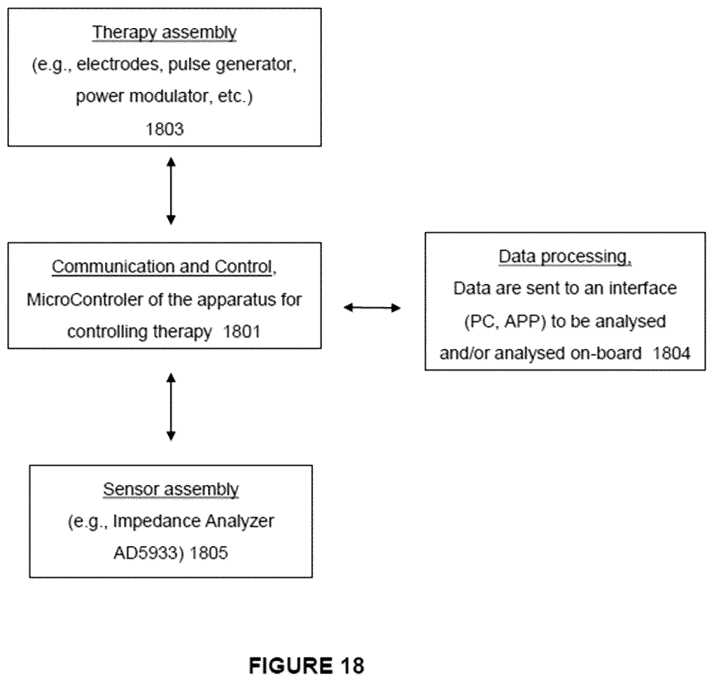

In any of these apparatuses, the sensing (e.g. the sensing assembly) and the therapeutic energy application of the apparatus may be integrated. For example, as schematically illustrated in FIG. 18, an apparatus may include one or more processors 1801, which may include a microcontroller that may coordinate the operation of the apparatus, including the application of therapy and/or the sensing of one or more biomarkers. The system may also include a plurality of electrodes for applying therapeutic energy to the oral muscle, as described above. The electrodes may be part of a therapy assembly or sub-system 1803 that may also include a pulse generator, power modulator, etc., which may be controlled by the communication and control assembly (e.g., controller) 1801. The apparatus may also include a sensor assembly 1805, as described herein. The sensor subassembly may also include one or more sensors (e.g., electrodes, thermistors, etc.) and/or may use the therapeutic electrodes. The apparatus may also include a data processing assembly 1804 which may be part of the controller or separate from the controller.

In operation, the methods and apparatuses described herein may be used to both sense a composition of one or more muscles (e.g., tongue) of the body, and may use the sensed composition to set, modify and/or track an applied therapeutic treatment. For example, an alternating electrical signal may be applied to a biological tissue such as the tongue to produce a complex electrical bioimpedance that is a function of tissue composition and applied signal frequencies. By studying the bioimpedance spectra of biological tissues over a wide range of frequencies, information regarding the morphology and anatomy of one or more tongue muscle can be determined.

For example, time-variable impedance over a range of frequencies linked to electrical conductivity of the genioglossus muscle may indicate the anatomical composition (including the structure and content) of the muscle, such as the type of muscle, or percent of, e.g., type 1 (slow endurance) and type 2 (fast twitch/power) muscle. This may correlate to the change in blood flow in the muscle resulting from a change in muscle structure over the course of a therapy. These apparatuses may also detect the overall volume change in the muscle (e.g., tongue), and/or the tongue fat content.

For example, by determining or monitoring the impedance of muscle of the tongue, based on bioimpedance analysis, before, during and/or after a treatment session or treatment plan, these method and apparatuses may verify the success of (and/or modify) the treatment. The change in bioimpedance may be communicated to the user, and/or to a physician, or caregiver, for example visually, aurally or otherwise.

Any of these methods and apparatuses may detect and monitor changes in muscle structure (morphology and anatomy) before, during and/or after applied therapy in order to modify the parameters to achieve the desired result. For example, these methods and apparatuses may determine or monitor the body composition of muscle tongue, based on bioimpedance analysis, before, during and/or after a treatment session or treatment plan.

In some cases, these methods and apparatuses may determine and/or monitor the bioimpedance of muscle (e.g., tongue) as a non-invasive tool for assessing the health of the muscle, such as the tongue, including in those with specific clinical conditions, such as neuromuscular conditions, and degenerative conditions, e.g., Amyotrophic Lateral Sclerosis (ALS), or other conditions, such as Type II Diabetes, etc. The combination of the sensing and therapeutic functions may be particularly helpful in these indications. For example, the detection and/or monitoring of the muscle composition, e.g., based on bioimpedance of the tongue) may be used as a non-invasive tool for muscle assessment, including in clinical conditions such as Oral Cancer (OSCC, Oral Squamous Cell Carcinoma/OPMDs, Oral Potentially Malignant Disorders, etc.).

It will also be appreciated by those skilled in the art that any number of combinations of the aforementioned features and/or those shown in the appended drawings provide clear advantages over the prior art and are therefore within the scope of the methods and apparatuses described herein described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the methods and apparatuses described herein. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present methods and apparatuses described herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the methods and apparatuses described herein as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the methods and apparatuses described herein as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

1.-32. (canceled)

33. An apparatus for determining a composition of one or more muscles of the mouth of a user, the apparatus comprising a mouthpiece including one or more electrodes for applying electrical stimulation to one or more muscles of the mouth of a user and a sensing assembly configured or configurable to determine or measure, in use, the composition of a tongue of the user.

34. The apparatus of claim 1, further comprising an impedance sensor.

35. The apparatus of claim 2, wherein the impedance sensor comprises a sensing assembly configured to evaluate the composition of the tongue at multiple frequencies up to 100 kHz.

36. The apparatus of claim 3, wherein the sensing assembly is configured to evaluate the tongue composition at a single frequency overtime.

37. The apparatus of claim 4, wherein the sensing assembly is configured to evaluate the tongue composition at multiple frequencies over time during a sampling window between 0 to 90 seconds or longer.

38. The apparatus of claim 2, wherein the impedance sensor comprises a plurality of impedance sensors.

39. The apparatus of claim 6, wherein the plurality of impedance sensors are configured to measure the impedance at different locations on the tongue of the user.

40. The apparatus of claim 6, wherein the plurality of impedance sensors are provided on different locations on the apparatus.

41. The apparatus of claim 6, wherein the plurality of impedance sensors are provided at different locations on the mouthpiece.

42. The apparatus of claim 2, wherein the sensor is selected from one or more of a temperature sensor, or an electromyography (EMG) sensor.

43. The apparatus of claim 1, wherein the sensing assembly is configured to determine or measure the tongue composition by determining or measuring a volumetric or mass ratio of muscle to fat of the tongue of the user.

44. The apparatus of claim 1, wherein the sensing assembly is configured to determine or measure the tongue composition by determining or measuring a total volume or mass of muscle and/or fat of the tongue of the user.

45. The apparatus of claim 1, wherein the sensing assembly is configured to determine or measure the tongue composition by determining or measuring a mass or volume of one or more types of muscle of the tongue of the user.

46. The apparatus of claim 1, wherein the sensing assembly is configured to determine or measure the tongue composition by determining or measuring a relative ratio of a first type of muscle to a second type of muscle of the tongue of the user.

47. The apparatus of claim 1, wherein the sensing assembly comprises an impedance converter configured or configurable to convert impedance data received from the sensor into user readable data.

48. The apparatus of claim 1, wherein the sensing assembly comprises a frequency adjuster or generator configured or configurable to adjust or generate a or the frequency of electrical energy supplied to the one or more electrodes.

49. The apparatus of claim 1, further wherein the mouthpiece comprises a pair of arms connected together at a connecting portion and extending away from one another, and/or the mouthpiece comprises flanges for overlying at least a portion of a dorsal or a sublingual surface of the user's tongue.

50. The apparatus of claim 1, further comprising a controller operable to alter electrical stimulation to the one or more muscles of the mouth of the user.

51. The apparatus of claim 1, further comprising memory to hold data relating to electrical stimulation applied to said one or more muscles of the user and/or data relating to an output of the sensing assembly.

52. A method of determining the tongue composition of a person, the method comprising;
locating one or more electrodes in a mouth of a person;
applying electrical stimulation via the one or more electrodes to one or more muscles of the mouth of the person; and
using a sensing assembly to determine the tongue composition of the person.
